SEQUENCE LISTING (191 sequences)

SEQ ID NO: 1 - DNA, 23 bp, Artificial sequence
  sense oligonucleotide primer for amplifying serine proteases
tgggtngtnacngcngcncaytg

SEQ ID NO: 2 - DNA, 20 bp, Artificial sequence
  antisense oligonucleotide primer for amplifying serine proteases
arnarngcnatntcnttncc

SEQ ID NO: 3 - DNA, 20 bp, Artificial sequence
  antisense oligonucleotide primer for amplifying serine proteases
arnggnccnccnswrtcncc

SEQ ID NO: 4 - DNA, 24 bp, Artificial sequence
  sense oligonucleotide primer for amplifying cysteine proteases
carggncartgyggnwsntgytgg

SEQ ID NO: 5 - DNA, 20 bp, Artificial sequence
  antisense oligonucleotide primer for amplifying cysteine proteases
tanccnccrttrcanccytc

SEQ ID NO: 6 - DNA, 20 bp, Artificial sequence
  sense oligonucleotide primer for amplifying metallo-proteases
ccnmgntgyggnrwnccnga

SEQ ID NO: 7 - DNA, 17 bp, Artificial sequence
  primer_bind: antisense oligonucleotide primer for amplifying metallo-proteases
ttrtgnccnanytcrtg

SEQ ID NO: 8 - DNA, 20 bp, Artificial sequence
  sense oligonucleotide primer specific for hepsin
tgtcccgatggcgagtgttt

SEQ ID NO: 9 - DNA, 20 bp, Artificial sequence
  antisense oligonucleotide primer specific for hepsin
cctgttggccatagtactgc

SEQ ID NO: 10 - DNA, 20 bp, Artificial sequence
  sense oligonucleotide primer specific for SCCE
agatgaatgagtacaccgtg

SEQ ID NO: 11 - DNA, 20 bp, Artificial sequence
  antisense oligonucleotide primer specific for SCCE
ccagtaagtccttgtaaacc

SEQ ID NO: 12 - DNA, 20 bp, Artificial sequence
  sense oligonucleotide primer specific for CompB
aagggacacgagagctgtat

SEQ ID NO: 13 - DNA, 20 bp, Artificial sequence
  antisense oligonucleotide primer specific for CompB
aagtggtagttggaggaagc

SEQ ID NO: 14 - DNA, 20 bp, Artificial sequence
  sense oligonucleotide primer specific for Cath-L
attggagagagaaaggctac

SEQ ID NO: 15 - DNA, 20 bp, Artificial sequence
  antisense oligonucleotide primer specific for Cath-L
cttgggattgtacttacagg

SEQ ID NO: 16 - DNA, 20 bp, Artificial sequence
  sense oligonucleotide primer specific for PUMP-1
cttccaaagtggtcacctac

SEQ ID NO: 17 - DNA, 20 bp, Artificial sequence
  antisense oligonucleotide primer specific for PUMP-1
ctagactgctaccatccgtc

SEQ ID NO: 18 - DNA, 17 bp, Artificial sequence
  sense oligonucleotide primer specific for b-tubulin
tgcattgacaacgaggc

SEQ ID NO: 19 - DNA, 17 bp, Artificial sequence
  antisense oligonucleotide primer specific for beta-tubulin
ctgtcttgacattgttg

SEQ ID NO: 20 - DNA, 20 bp, Artificial sequence
  sense oligonucleotide primer specific for Protease M
ctgtgatccaccctgactat

SEQ ID NO: 21 - DNA, 20 bp, Artificial sequence
  antisense oligonucleotide primer specific for Protease M
caggtggatgtatgcacact

SEQ ID NO: 22 - DNA, 20 bp, Artificial sequence
  sense oligonucleotide primer specific for TADG-12
gcgcactgtgtttatgagat

SEQ ID NO: 23 - DNA, 20 bp, Artificial sequence
  antisense oligonucleotide primer specific for TADG-12
ctctttggcttgtacttgct

SEQ ID NO: 24 - DNA, 20 bp, Artificial sequence
  sense oligonucleotide primer specific for TADG-13
tgagggacatcattatgcac

SEQ ID NO: 25 - DNA, 20 bp, Artificial sequence
  antisense oligonucleotide primer specific for TADG-13
caagttttccccataattgg

SEQ ID NO: 26 - DNA, 20 bp, Artificial sequence
  sense oligonucleotide primer specific for TADG-14
acagtacgcctgggagacca

SEQ ID NO: 27 - DNA, 20 bp, Artificial sequence
  antisense oligonucleotide primer specific for TADG-14
ctgagacggtgcaattctgg

SEQ ID NO: 28 - protein, 9 aa, Homo sapiens
  Residues 170-178 of the hepsin protein
SLGRWPWQV

SEQ ID NO: 29 - protein, 9 aa, Homo sapiens
  Residues 191-199 of the hepsin protein
SLLSGDWVL

SEQ ID NO: 30 - protein, 9 aa, Homo sapiens
  Residues 229-237 of the hepsin protein
GLQLGVQAV

SEQ ID NO: 31 - protein, 9 aa, Homo sapiens
  Residues 392-400 of the hepsin protein
KVSDFREWI

SEQ ID NO: 32 - protein, 9 aa, Homo sapiens
  Residues 308-316 of the hepsin protein
VLQEARVPI

SEQ ID NO: 33 - protein, 9 aa, Homo sapiens
  Residues 130-138 of the hepsin protein
RLLEVISVC

SEQ ID NO: 34 - protein, 9 aa, Homo sapiens
  Residues 98-106 of the hepsin protein
ALTHSELDV

SEQ ID NO: 35 - protein, 9 aa, Homo sapiens
  Residues 211-219 of the hepsin protein
VLSRWRVFA

SEQ ID NO: 36 - protein, 9 aa, Homo sapiens
  Residues 26-34 of the hepsin protein
LLLLTAIGA

SEQ ID NO: 37 - protein, 9 aa, Homo sapiens
  Residues 284-292 of the hepsin protein
ALVDGKICT

SEQ ID NO: 38 - protein, 9 aa, Homo sapiens
  Residues 145-153 of the hepsin protein
FLAAICQDC

SEQ ID NO: 39 - protein, 9 aa, Homo sapiens
  Residues 192-200 of the hepsin protein
LLSGDWVLT

SEQ ID NO: 40 - protein, 9 aa, Homo sapiens
  Residues 20-28 of the hepsin protein
ALTAGTLLL

SEQ ID NO: 41 - protein, 9 aa, Homo sapiens
  Residues 259-267 of the hepsin protein
ALVHLSSPL

SEQ ID NO: 42 - protein, 9 aa, Homo sapiens
  Residues 277-285 of the hepsin protein
CLPAAGQAL

SEQ ID NO: 43 - protein, 9 aa, Homo sapiens
  Residues 230-238 of the hepsin protein
LQLGVQAVV

SEQ ID NO: 44 - protein, 9 aa, Homo sapiens
  Residues 268-276 of the hepsin protein
PLTEYIQPV

SEQ ID NO: 45 - protein, 9 aa, Homo sapiens
  Residues 31-39 of the hepsin protein
AIGAASWAI

SEQ ID NO: 46 - protein, 9 aa, Homo sapiens
  Residues 285-293 of the hepsin protein
LVDGKICTV

SEQ ID NO: 47 - protein, 9 aa, Homo sapiens
  Residues 27-35 of the hepsin protein
LLLTAIGAA

SEQ ID NO: 48 - protein, 9 aa, Homo sapiens
  Residues 191-199 of the hepsin protein
SLLSGDWVL

SEQ ID NO: 49 - protein, 9 aa, Homo sapiens
  Residues 163-171 of the hepsin protein
IVGGRDTSL

SEQ ID NO: 50 - protein, 9 aa, Homo sapiens
  Residues 392-400 of the hepsin protein
KVSDFREWI

SEQ ID NO: 51 - protein, 9 aa, Homo sapiens
  Residues 64-72 of the hepsin protein
MVFDKTEGT

SEQ ID NO: 52 - protein, 9 aa, Homo sapiens
  Residues 236-244 of the hepsin protein
AVVYHGGYL

SEQ ID NO: 53 - protein, 9 aa, Homo sapiens
  Residues 55-63 of the hepsin protein
QVSSADARL

SEQ ID NO: 54 - protein, 9 aa, Homo sapiens
  Residues 130-138 of the hepsin protein
RLLEVISVC

SEQ ID NO: 55 - protein, 9 aa, Homo sapiens
  Residues 230-238 of the hepsin protein
LQLGVQAVV

SEQ ID NO: 56 - protein, 9 aa, Homo sapiens
  Residues 20-28 of the hepsin protein
ALTAGTLLL

SEQ ID NO: 57 - protein, 9 aa, Homo sapiens
  Residues 259-267 of the hepsin protein
ALVHLSSPL

SEQ ID NO: 58 - protein, 9 aa, Homo sapiens
  Residues 277-285 of the hepsin protein
CLPAAGQAL

SEQ ID NO: 59 - protein, 9 aa, Homo sapiens
  Residues 17-25 of the hepsin protein
KVAALTAGT

SEQ ID NO: 60 - protein, 9 aa, Homo sapiens
  Residues 285-293 of the hepsin protein
LVDGKICTV

SEQ ID NO: 61 - protein, 9 aa, Homo sapiens
  Residues 308-316 of the hepsin protein
VLQEARVPI

SEQ ID NO: 62 - protein, 9 aa, Homo sapiens
  Residues 27-35 of the hepsin protein
LLLTAIGAA

SEQ ID NO: 63 - protein, 9 aa, Homo sapiens
  Residues 229-237 of the hepsin protein
GLQLGVQAV

SEQ ID NO: 64 - protein, 9 aa, Homo sapiens
  Residues 313-321 of the hepsin protein
RVPIISNDV

SEQ ID NO: 65 - protein, 9 aa, Homo sapiens
  Residues 88-96 of the hepsin protein
LSCEEMGFL

SEQ ID NO: 66 - protein, 9 aa, Homo sapiens
  Residues 192-200 of the hepsin protein
LLSGDWVLT

SEQ ID NO: 67 - protein, 9 aa, Homo sapiens
  Residues 284-292 of the hepsin protein
ALVDGKICT

SEQ ID NO: 68 - protein, 9 aa, Homo sapiens
  Residues 89-97 of the hepsin protein
SCEEMGFLR

SEQ ID NO: 69 - protein, 9 aa, Homo sapiens
  Residues 58-66 of the hepsin protein
SADARLMVF

SEQ ID NO: 70 - protein, 9 aa, Homo sapiens
  Residues 393-401 of the hepsin protein
VSDFREWIF

SEQ ID NO: 71 - protein, 9 aa, Homo sapiens
  Residues 407-415 of the hepsin protein
HSEASGMVT

SEQ ID NO: 72 - protein, 9 aa, Homo sapiens
  Residues 137-145 of the hepsin protein
VCDCPRGRF

SEQ ID NO: 73 - protein, 9 aa, Homo sapiens
  Residues 269-277 of the hepsin protein
LTEYIQPVC

SEQ ID NO: 74 - protein, 9 aa, Homo sapiens
  Residues 47-55 of the hepsin protein
DQEPLYPVQ

SEQ ID NO: 75 - protein, 9 aa, Homo sapiens
  Residues 119-127 of the hepsin protein
CVDEGRLPH

SEQ ID NO: 76 - protein, 9 aa, Homo sapiens
  Residues 68-76 of the hepsin protein
KTEGTWRLL

SEQ ID NO: 77 - protein, 9 aa, Homo sapiens
  Residues 101-109 of the hepsin protein
HSELDVRTA

SEQ ID NO: 78 - protein, 9 aa, Homo sapiens
  Residues 250-258 of the hepsin protein
NSEENSNDI

SEQ ID NO: 79 - protein, 9 aa, Homo sapiens
  Residues 293-301 of the hepsin protein
VTGWGNTQY

SEQ ID NO: 80 - protein, 9 aa, Homo sapiens
  Residues 231-239 of the hepsin protein
QLGVQAVVY

SEQ ID NO: 81 - protein, 9 aa, Homo sapiens
  Residues 103-111 of the hepsin protein
ELDVRTAGA

SEQ ID NO: 82 - protein, 9 aa, Homo sapiens
  Residues 378-386 of the hepsin protein
GTGCALAQK

SEQ ID NO: 83 - protein, 9 aa, Homo sapiens
  Residues 358-366 of the hepsin protein
VCEDSISRT

SEQ ID NO: 84 - protein, 9 aa, Homo sapiens
  Residues 264-272 of the hepsin protein
SSPLPLTEY

SEQ ID NO: 85 - protein, 9 aa, Homo sapiens
  Residues 87-95 of the hepsin protein
GLSCEEMGF

SEQ ID NO: 86 - protein, 9 aa, Homo sapiens
  Residues 272-280 of the hepsin protein
YIQPVCLPA

SEQ ID NO: 87 - protein, 9 aa, Homo sapiens
  Residues 345-353 of the hepsin protein
GIDACQGDS

SEQ ID NO: 88 - protein, 9 aa, Homo sapiens
  Residues 301-309 of the hepsin protein
YYGQQAGVL

SEQ ID NO: 89 - protein, 9 aa, Homo sapiens
  Residues 238-246 of the hepsin protein
VYHGGYLPF

SEQ ID NO: 90 - protein, 9 aa, Homo sapiens
  Residues 204-212 of the hepsin protein
CFPERNRVL

SEQ ID NO: 91 - protein, 9 aa, Homo sapiens
  Residues 117-125 of the hepsin protein
FFCVDEGRL

SEQ ID NO: 92 - protein, 9 aa, Homo sapiens
  Residues 124-132 of the hepsin protein
RLPHTQRLL

SEQ ID NO: 93 - protein, 9 aa, Homo sapiens
  Residues 80-88 of the hepsin protein
RSNARVAGL

SEQ ID NO: 94 - protein, 9 aa, Homo sapiens
  Residues 68-76 of the hepsin protein
KTEGTWRLL

SEQ ID NO: 95 - protein, 9 aa, Homo sapiens
  Residues 340-348 of the hepsin protein
GYPEGGIDA

SEQ ID NO: 96 - protein, 9 aa, Homo sapiens
  Residues 242-250 of the hepsin protein
GYLPFRDPN

SEQ ID NO: 97 - protein, 9 aa, Homo sapiens
  Residues 51-59 of the hepsin protein
LYPVQVSSA

SEQ ID NO: 98 - protein, 9 aa, Homo sapiens
  Residues 259-267 of the hepsin protein
ALVHLSSPL

SEQ ID NO: 99 - protein, 9 aa, Homo sapiens
  Residues 277-285 of the hepsin protein
CLPAAGQAL

SEQ ID NO: 100 - protein, 9 aa, Homo sapiens
  Residues 191-199 of the hepsin protein
SLLSGDWVL

SEQ ID NO: 101 - protein, 9 aa, Homo sapiens
  Residues 210-218 of the hepsin protein
RVLSRWRVF

SEQ ID NO: 102 - protein, 9 aa, Homo sapiens
  Residues 222-230 of the hepsin protein
VAQASPHGL

SEQ ID NO: 103 - protein, 9 aa, Homo sapiens
  Residues 236-244 of the hepsin protein
AVVYHGGYL

SEQ ID NO: 104 - protein, 9 aa, Homo sapiens
  Residues 19-27 of the hepsin protein
AALTAGTLL

SEQ ID NO: 105 - protein, 9 aa, Homo sapiens
  Residues 36-44 of the hepsin protein
SWAIVAVLL

SEQ ID NO: 106 - protein, 9 aa, Homo sapiens
  Residues 35-43 of the hepsin protein
ASWAIVAVL

SEQ ID NO: 107 - protein, 9 aa, Homo sapiens
  Residues 300-308 of the hepsin protein
QYYGQQAGV

SEQ ID NO: 108 - protein, 9 aa, Homo sapiens
  Residues 363-371 of the hepsin protein
ISRTPRWRL

SEQ ID NO: 109 - protein, 9 aa, Homo sapiens
  Residues 366-374 of the hepsin protein
TPRWRLCGI

SEQ ID NO: 110 - protein, 9 aa, Homo sapiens
  Residues 236-244 of the hepsin protein
AVVYHGGYL

SEQ ID NO: 111 - protein, 9 aa, Homo sapiens
  Residues 13-21 of the hepsin protein
CSRPKVAAL

SEQ ID NO: 112 - protein, 9 aa, Homo sapiens
  Residues 179-187 of the hepsin protein
SLRYDGAHL

SEQ ID NO: 113 - protein, 9 aa, Homo sapiens
  Residues 43-51 of the hepsin protein
LLRSDQEPL

SEQ ID NO: 114 - protein, 9 aa, Homo sapiens
  Residues 19-27 of the hepsin protein
AALTAGTLL

SEQ ID NO: 115 - protein, 9 aa, Homo sapiens
  Residues 55-63 of the hepsin protein
QVSSADARL

SEQ ID NO: 116 - protein, 9 aa, Homo sapiens
  Residues 163-171 of the hepsin protein
IVGGRDTSL

SEQ ID NO: 117 - protein, 9 aa, Homo sapiens
  Residues 140-148 of the hepsin protein
CPRGRFLAA

SEQ ID NO: 118 - protein, 9 aa, Homo sapiens
  Residues 20-28 of the hepsin protein
ALTAGTLLL

SEQ ID NO: 119 - protein, 9 aa, Homo sapiens
  Residues 409-417 of the hepsin protein
EASGMVTQL

SEQ ID NO: 120 - protein, 9 aa, Homo sapiens
  Residues 259-267 of the hepsin protein
ALVHLSSPL

SEQ ID NO: 121 - protein, 9 aa, Homo sapiens
  Residues 35-43 of the hepsin protein
ASWAIVAVL

SEQ ID NO: 122 - protein, 9 aa, Homo sapiens
  Residues 184-192 of the hepsin protein
GAHLCGGSL

SEQ ID NO: 123 - protein, 9 aa, Homo sapiens
  Residues 18-26 of the hepsin protein
VAALTAGTL

SEQ ID NO: 124 - protein, 9 aa, Homo sapiens
  Residues 222-230 of the hepsin protein
VAQASPHGL

SEQ ID NO: 125 - protein, 9 aa, Homo sapiens
  Residues 224-232 of the hepsin protein
QASPHGLQL

SEQ ID NO: 126 - protein, 9 aa, Homo sapiens
  Residues 265-273 of the hepsin protein
SPLPLTEYI

SEQ ID NO: 127 - protein, 9 aa, Homo sapiens
  Residues 355-363 of the hepsin protein
GPFVCEDSI

SEQ ID NO: 128 - protein, 9 aa, Homo sapiens
  Residues 13-21 of the hepsin protein
CSRPKVAAL

SEQ ID NO: 129 - protein, 9 aa, Homo sapiens
  Residues 366-374 of the hepsin protein
TPRWRLCGI

SEQ ID NO: 130 - protein, 9 aa, Homo sapiens
  Residues 140-148 of the hepsin protein
CPRGRFLAA

SEQ ID NO: 131 - protein, 9 aa, Homo sapiens
  Residues 152-160 of the hepsin protein
DCGRRKLPV

SEQ ID NO: 132 - protein, 9 aa, Homo sapiens
  Residues 363-371 of the hepsin protein
ISRTPRWRL

SEQ ID NO: 133 - protein, 9 aa, Homo sapiens
  Residues 133-141 of the hepsin protein
IVGGRDTSL

SEQ ID NO: 134 - protein, 9 aa, Homo sapiens
  Residues 331-339 of the hepsin protein
QIKPKMFCA

SEQ ID NO: 135 - protein, 9 aa, Homo sapiens
  Residues 80-88 of the hepsin protein
RSNARVAGL

SEQ ID NO: 136 - protein, 9 aa, Homo sapiens
  Residues 179-187 of the hepsin protein
SLRYDGAHL

SEQ ID NO: 137 - protein, 9 aa, Homo sapiens
  Residues 43-51 of the hepsin protein
LLRSDQEPL

SEQ ID NO: 138 - protein, 9 aa, Homo sapiens
  Residues 409-417 of the hepsin protein
EASGMVTQL

SEQ ID NO: 139 - protein, 9 aa, Homo sapiens
  Residues 311-319 of the hepsin protein
EARVPIISN

SEQ ID NO: 140 - protein, 9 aa, Homo sapiens
  Residues 222-230 of the hepsin protein
VAQASPHGL

SEQ ID NO: 141 - protein, 9 aa, Homo sapiens
  Residues 19-27 of the hepsin protein
AALTAGTLL

SEQ ID NO: 142 - protein, 9 aa, Homo sapiens
  Residues 18-26 of the hepsin protein
VAALTAGTL

SEQ ID NO: 143 - protein, 9 aa, Homo sapiens
  Residues 184-192 of the hepsin protein
GAHLCGGSL

SEQ ID NO: 144 - protein, 9 aa, Homo sapiens
  Residues 224-232 of the hepsin protein
QASPHGLQL

SEQ ID NO: 145 - protein, 9 aa, Homo sapiens
  Residues 82-90 of the hepsin protein
NARVAGLSC

SEQ ID NO: 146 - protein, 9 aa, Homo sapiens
  Residues 204-212 of the hepsin protein
CFPERNRVL

SEQ ID NO: 147 - protein, 9 aa, Homo sapiens
  Residues 212-220 of the hepsin protein
LSRWRVFAG

SEQ ID NO: 148 - protein, 9 aa, Homo sapiens
  Residues 172-180 of the hepsin protein
GRWPWQVSL

SEQ ID NO: 149 - protein, 9 aa, Homo sapiens
  Residues 44-52 of the hepsin protein
LRSDQEPLY

SEQ ID NO: 150 - protein, 9 aa, Homo sapiens
  Residues 155-163 of the hepsin protein
RRKLPVDRI

SEQ ID NO: 151 - protein, 9 aa, Homo sapiens
  Residues 213-221 of the hepsin protein
SRWRVFAGA

SEQ ID NO: 152 - protein, 9 aa, Homo sapiens
  Residues 166-174 of the hepsin protein
GRDTSLGRW

SEQ ID NO: 153 - protein, 9 aa, Homo sapiens
  Residues 369-377 of the hepsin protein
WRLCGIVSW

SEQ ID NO: 154 - protein, 9 aa, Homo sapiens
  Residues 180-188 of the hepsin protein
LRYDGAHLC

SEQ ID NO: 155 - protein, 9 aa, Homo sapiens
  Residues 96-104 of the hepsin protein
LRALTHSEL

SEQ ID NO: 156 - protein, 9 aa, Homo sapiens
  Residues 396-404 of the hepsin protein
FREWIFQAI

SEQ ID NO: 157 - protein, 9 aa, Homo sapiens
  Residues 123-131 of the hepsin protein
GRLPHTQRL

SEQ ID NO: 158 - protein, 9 aa, Homo sapiens
  Residues 207-215 of the hepsin protein
ERNRVLSRW

SEQ ID NO: 159 - protein, 9 aa, Homo sapiens
  Residues 209-217 of the hepsin protein
NRVLSRWRV

SEQ ID NO: 160 - protein, 9 aa, Homo sapiens
  Residues 14-22 of the hepsin protein
SRPKVAALT

SEQ ID NO: 161 - protein, 9 aa, Homo sapiens
  Residues 106-114 of the hepsin protein
VRTAGANGT

SEQ ID NO: 162 - protein, 9 aa, Homo sapiens
  Residues 129-137 of the hepsin protein
QRLLEVISV

SEQ ID NO: 163 - protein, 9 aa, Homo sapiens
  Residues 349-357 of the hepsin protein
CQGDSGGPF

SEQ ID NO: 164 - protein, 9 aa, Homo sapiens
  Residues 61-69 of the hepsin protein
ARLMVFDKT

SEQ ID NO: 165 - protein, 9 aa, Homo sapiens
  Residues 215-223 of the hepsin protein
WRVFAGAVA

SEQ ID NO: 166 - protein, 9 aa, Homo sapiens
  Residues 143-151 of the hepsin protein
GRFLAAICQ

SEQ ID NO: 167 - protein, 9 aa, Homo sapiens
  Residues 246-254 of the hepsin protein
FRDPNSEEN

SEQ ID NO: 168 - protein, 9 aa, Homo sapiens
  Residues 132-140 of the hepsin protein
LEVISVCDC

SEQ ID NO: 169 - protein, 9 aa, Homo sapiens
  Residues 91-99 of the hepsin protein
EEMGFLRAL

SEQ ID NO: 170 - protein, 9 aa, Homo sapiens
  Residues 264-272 of the hepsin protein
SSPLPLTEY

SEQ ID NO: 171 - protein, 9 aa, Homo sapiens
  Residues 310-318 of the hepsin protein
QEARVPIIS

SEQ ID NO: 172 - protein, 9 aa, Homo sapiens
  Residues 319-327 of the hepsin protein
NDVCNGADF

SEQ ID NO: 173 - protein, 9 aa, Homo sapiens
  Residues 4-12 of the hepsin protein
KEGGRTVPC

SEQ ID NO: 174 - protein, 9 aa, Homo sapiens
  Residues 251-259 of the hepsin protein
SEENSNDIA

SEQ ID NO: 175 - protein, 9 aa, Homo sapiens
  Residues 256-264 of the hepsin protein
NDIALVHLS

SEQ ID NO: 176 - protein, 9 aa, Homo sapiens
  Residues 294-302 of the hepsin protein
TGWGNTQYY

SEQ ID NO: 177 - protein, 9 aa, Homo sapiens
  Residues 361-369 of the hepsin protein
DSISRTPRW

SEQ ID NO: 178 - protein, 9 aa, Homo sapiens
  Residues 235-243 of the hepsin protein
QAVVYHGGY

SEQ ID NO: 179 - protein, 9 aa, Homo sapiens
  Residues 109-117 of the hepsin protein
AGANGTSGF

SEQ ID NO: 180 - protein, 9 aa, Homo sapiens
  Residues 270-278 of the hepsin protein
TEYIQPVCL

SEQ ID NO: 181 - protein, 9 aa, Homo sapiens
  Residues 174-182 of the hepsin protein
WPWQVSLRY

SEQ ID NO: 182 - protein, 9 aa, Homo sapiens
  Residues 293-301 of the hepsin protein
VTGWGNTQY

SEQ ID NO: 183 - protein, 9 aa, Homo sapiens
  Residues 69-77 of the hepsin protein
TEGTWRLLC

SEQ ID NO: 184 - protein, 9 aa, Homo sapiens
  Residues 90-98 of the hepsin protein
CEEMGFLRA

SEQ ID NO: 185 - protein, 9 aa, Homo sapiens
  Residues 252-260 of the hepsin protein
EENSNDIAL

SEQ ID NO: 186 - protein, 9 aa, Homo sapiens
  Residues 48-56 of the hepsin protein
QEPLYPVQV

SEQ ID NO: 187 - protein, 9 aa, Homo sapiens
  Residues 102-110 of the hepsin protein
SELDVRTAG

SEQ ID NO: 188 - DNA, 1783 bp, Homo sapiens
  full length cDNA of hepsin
tcgagcccgctttccagggaccctacctgagggcccacaggtgaggcagcctggcctagcaggccccacgccaccgcctctgcctccaggccgcccgctgctgcggggccaccatgctcctgcccaggcctggagactgacccgaccccggcactacctcgaggctccgcccccacctgctggaccccagggtcccaccctggcccaggaggtcagccagggaatcattaacaagaggcagtgacatggcgcagaaggagggtggccggactgtgccatgctgctccagacccaaggtggcagctctcactgcggggaccctgctacttctgacagccatcggggcggcatcctgggccattgtggctgttctcctcaggagtgaccaggagccgctgtacccagtgcaggtcagctctgcggacgctcggctcatggtctttgacaagacggaagggacgtggcggctgctgtgctcctcgcgctccaacgccagggtagccggactcagctgcgaggagatgggcttcctcagggcactgacccactccgagctggacgtgcgaacggcgggcgccaatggcacgtcgggcttcttctgtgtggacgaggggaggctgccccacacccagaggctgctggaggtcatctccgtgtgtgattgccccagaggccgtttcttggccgccatctgccaagactgtggccgcaggaagctgcccgtggaccgcatcgtgggaggccgggacaccagcttgggccggtggccgtggcaagtcagccttcgctatgatggagcacacctctgtgggggatccctgctctccggggactgggtgctgacagccgcccactgcttcccggagcggaaccgggtcctgtcccgatggcgagtgtttgccggtgccgtggcccaggcctctccccacggtctgcagctgggggtgcaggctgtggtctaccacgggggctatcttccctttcgggaccccaacagcgaggagaacagcaacgatattgccctggtccacctctccagtcccctgcccctcacagaatacatccagcctgtgtgcctcccagctgccggccaggccctggtggatggcaagatctgtaccgtgacgggctggggcaacacgcagtactatggccaacaggccggggtactccaggaggctcgagtccccataatcagcaatgatgtctgcaatggcgctgacttctatggaaaccagatcaagcccaagatgttctgtgctggctaccccgagggtggcattgatgcctgccagggcgacagcggtggtccctttgtgtgtgaggacagcatctctcggacgccacgttggcggctgtgtggcattgtgagttggggcactggctgtgccctggcccagaagccaggcgtctacaccaaagtcagtgacttccgggagtggatcttccaggccataaagactcactccgaagccagcggcatggtgacccagctctgaccggtggcttctcgctgcgcagcctccagggcccgaggtgatcccggtggtgggatccacgctgggccgaggatgggacgtttttcttcttgggcccggtccacaggtccaaggacaccctccctccagggtcctctcttccacagtggcgggcccactcagccccgagaccacccaacctcaccctcctgacccccatgtaaatattgttctgctgtctgggactcctgtctaggtgcccctgatgatgggatgctctttaaataataaagatggttttgatt

SEQ ID NO: 189 - protein, 10 aa, Homo sapiens
  Residues 42-51 of the hepsin protein
VLLRSDICTV

SEQ ID NO: 190 - protein, 10 aa, Homo sapiens
  Residues 284-293 of the hepsin protein
ALVDGKICTV

SEQ ID NO: 191 - protein, 10 aa, Homo sapiens
  Residues 308-317 of the hepsin protein
VLQEARVPII

(19) United States
(12) Patent Application Publication
(10) Pub. No.: US 2011/0213337 A1
(43) Pub. Date: Sep. 1, 2011

(54) Methods for the early diagnosis of ovarian cancer

(76) Inventors: Timothy J. O'Brien, Little Rock, AR (US); Martin J. Cannon, Little Rock, AR (US); Alessandro Santin, Little Rock, AR (US)

(21) Appl. No.: 13/066,897

(22) Filed: Apr. 27, 2011

Related U.S. Application Data
(63) Continuation-in-part of application No. 11/978,259, filed on Oct. 29, 2007, now Pat. No. 7,939,321.

(51) Int. Cl. A61M 5/00; C12N 5/078; C12N 15/85; C12Q 1/37; A61K 39/00; C12N 5/0781; C12N 5/0783; C12N 5/0784; C07H 21/04; A61K 31/7088; A61P 37/04; A61P 35/00

(52) U.S. Cl. 604/522; 435/375; 435/331; 435/455; 435/23; 424/185.1; 536/23.2; 514/44 R

(57) ABSTRACT

The disclosed nucleic acid primer sets, used in combination with quantitative amplification (PCR) of tissue cDNA, can indicate the presence of specific proteases in a tissue sample. Specifically, the present invention relates to expression of hepsin protease. The detected proteases are themselves specifically over-expressed in certain cancers, and the presence of their genetic precursors may serve for early detection of associated ovarian and other malignancies, and for the design of interactive therapies for cancer treatment.

### CROSS-REFERENCE TO RELATED APPLICATIONS

This is a divisional application of pending continuation-in-part application U.S. Ser. No. 11/978,259, which claims the benefit of priority under 35 USC §120 of U.S. Ser. No. 10/102,283, filed Mar. 20, 2002, now U.S. Pat. No. 6,875,609, which claims benefit of priority under 35 USC §120 of continuation-in-part application U.S. Ser. No. 09/919,048, filed Jul. 30, 2001, now U.S. Pat. No. 6,787,354, which claims benefit of priority under 35 USC §120 of divisional application U.S. Ser. No. 09/861,966, filed May 21, 2001, now U.S. Pat. No. 6,518,028, which claims benefit of priority under 35 USC §120 of continuation-in-part application U.S. Ser. No. 09/510,738, filed Feb. 22, 2000, now U.S. Pat. No. 6,268,165, which claims benefit of priority under 35 USC §120 of nonprovisional application U.S. Ser. No. 09/039,211, filed Mar. 14, 1998, now U.S. Pat. No. 6,303,318, which claims benefit of priority under 35 USC §119(e) of provisional application U.S. Ser. No. 60/041,404, filed Mar. 19, 1997, now abandoned, the entirety of all of which hereby are incorporated by reference.

### BACKGROUND OF THE INVENTION

1. Field of the Invention

Generally, the present invention relates to the fields of molecular biology and medicine. More specifically, the present invention is in the field of cancer research, especially ovarian cancer diagnosis.

2. Background of the Invention

In order for malignant cells to grow, spread or metastasize, they must have the capacity to invade local host tissue, dissociate or shed from the primary tumor, enter and survive in the bloodstream, implant by invasion into the surface of the target organ and establish an environment conducive for new colony growth, including the induction of angiogenic and growth factors. During this progression, natural tissue barriers such as basement membranes and connective tissue have to be degraded. These barriers include collagen, laminin, fibronectin, proteoglycans and extracellular matrix glycoproteins. Degradation of these natural barriers, both those surrounding the primary tumor and at the sites of metastatic invasion, is believed to be brought about by the action of a matrix of extracellular proteases.

Proteases have been classified into four families: serine proteases, metallo-proteases, aspartic proteases and cysteine proteases. Many proteases have been shown to be involved in human disease processes and these enzymes are targets for the development of inhibitors as new therapeutic agents. Certain individual proteases are induced and overexpressed in a diverse group of cancers, and as such, are potential candidates for markers of early diagnosis and targets for possible therapeutic intervention. A group of examples are shown in Table 1.

TABLE 1
Known Proteases Expressed In Various Cancers

| | Gastric | Brain | Breast | Ovarian |
|---|---|---|---|---|
| Serine | uPA | uPA | NES-1 | NES-1 |
| Proteases: | PAI-1 | PAI-1 | uPA | uPA |
| | | tPA | | PAI-2 |
| Cysteine | Cathepsin B | Cathepsin L | Cathepsin B | Cathepsin B |
| Proteases: | Cathepsin L | Cathepsin L | Cathepsin L | |
| Metallo- | Matrilysin* | Matrilysin | Stromelysin-3 | MMP-2 |
| proteases: | Collagenase* | Stromelysin | MMP-8 | |
| | Stromelysin-I* | Gelatinase B | MMP-9 | |
| | | | Gelatinase A | |

uPA, Urokinase-type plasminogen activator; tPA, Tissue-type plasminogen activator; PAI-I, Plasminogen activator 0 inhibitors; PAI-2, Plasminogen activator inhibitors; NES-1, Normal epithelial cell-specific-1; MMP, Matrix P metallo-protease.
*Overexpressed in gastrointestinal ulcers.

There is a good body of evidence supporting the downregulation or inhibition of individual proteases and the reduction in invasive capacity or malignancy. In work by Clark et al., inhibition of in vitro growth of human small cell lung cancer was demonstrated using a general serine protease inhibitor. More recently, Torres-Rosedo et al., (1) demonstrated an inhibition of hepatoma tumor cell growth using specific antisense inhibitors for the serine protease hepsin gene. Metastatic potential of melanoma cells has also been shown to be reduced in a mouse model using a synthetic inhibitor (batimastat) of metallo-proteases. Powell et al. (2) presented evidence to confirm that the expression of extracellular proteases in a non-metastatic prostate cancer cell line enhances their malignant progression. Specifically, enhanced metastasis was demonstrated after introducing and expressing the PUMP-1 metallo-protease gene. There is also a body of data to support the notion that expression of cell surface proteases on relatively non-metastatic cell types increases the invasive potential of such cells.

To date, ovarian cancer remains the number one killer of women with gynecologic malignant hyperplasia. Approximately 75% of women diagnosed with such cancers are already at an advanced stage (III and IV) of the disease at their initial diagnosis. During the past 20 years, neither diagnosis nor five-year survival rates have greatly improved for these patients. This is substantially due to the high percentage of high-stage initial detection of the disease. Therefore, the challenge remains to develop new markers that improve early diagnosis and thereby reduce the percentage of high-stage initial diagnoses. The ability to disengage from one tissue and re-engage the surface of another tissue is what provides for the morbidity and mortality associated with this disease. Therefore, extracellular proteases may be good candidates for markers of malignant ovarian hyperplasia.

Thus, the prior art is deficient in a tumor marker useful as an indicator of early disease, particularly for ovarian cancers. The present invention fulfills this long-standing need and desire in the art.

### SUMMARY OF THE INVENTION

This invention allows for the detection of cancer, especially ovarian cancer, by screening for hepsin mRNA in tissue, which is indicative of the hepsin protease, which is shown herein to be specifically associated with the surface of 80 percent of ovarian and other tumors. Proteases are considered to be an integral part of tumor growth and metastasis, and therefore, markers indicative of their presence or absence are useful for the diagnosis of cancer. Furthermore, the present invention is useful for treatment, i.e., by inhibiting hepsin or expression of hepsin, for targeted therapy, for vaccination, etc.

In one embodiment of the present invention, there is provided a method for detecting malignant hyperplasia in a biological sample by detecting hepsin mRNA in the sample. The presence of the hepsin mRNA in the sample is indicative of the presence of malignant hyperplasia, and the absence of the hepsin mRNA in the sample is indicative of the absence of malignant hyperplasia.

In another embodiment of the present invention, there are provided methods of inhibiting expression of hepsin in a cell by introducing into a cell a vector encoding an antisense hepsin mRNA or an antibody that binds the hepsin protein.

In yet another embodiment of the present invention, there is provided a method of targeted therapy to an individual, comprising the step of administering a compound to an individual, wherein the compound has a targeting moiety and a therapeutic moiety, wherein the targeting moiety is specific for hepsin.

In still yet another embodiment of the present invention, there are provided methods of vaccinating an individual against hepsin or produce immune-activated cells directed toward hepsin by inoculating an individual with an expression vector encoding a hepsin protein or a fragment thereof.

The present invention also provides methods of immunotherapy targeted toward hepsin in an individual, involving the steps of generating dendritic cells in vitro from peripheral blood drawn from an individual, loading these dendritic cells with hepsin protein or a fragment thereof, then transferring these dendritic cells back to the individual in single or multiple doses. Hepsin-loaded or hepsin-expressing dendritic cells can also be used to stimulate hepsin-specific T cell responses in vitro, followed by adoptive immunotherapy in which the individual is given autologous hepsin-specific T cells.

In another embodiment of the present invention, there are provided compositions comprising immunogenic fragments of hepsin protein or an oligonucleotide having a sequence complementary to SEQ ID NO:188. Also embodied is a method of treating a neoplastic state in an individual in need of such treatment with an effective dose of the above-described oligonucleotide.

In another embodiment of the present invention, there is provided a method of screening for compounds that inhibit hepsin activity, comprising the steps of contacting a sample with a compound, wherein the sample comprises hepsin protein; and assaying for hepsin protease activity. A decrease in the hepsin protease activity in the presence of the compound relative to hepsin protease activity in the absence of the compound is indicative of a compound that inhibits hepsin activity.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

The appended drawings have been included herein so that the above-recited features, advantages and objects of the invention will become clear and can be understood in detail. These drawings form a part of the specification. It is to be noted, however, that the appended drawings illustrate preferred embodiments of the invention and should not be considered to limit the scope of the invention.

FIG. 1 shows agarose gel comparison of PCR products derived from normal and carcinoma cDNA.

FIG. 2 shows Northern blot analysis of ovarian tumors using hepsin, SCCE, PUMP-1, TADG-14 and β-tubulin probes.

FIG. 3 shows amplification with serine protease redundant primers: histidine sense (S1) with aspartic acid antisense (AS1), using normal cDNA (Lane 1) and tumor cDNA (Lane 2); and histidine sense (S1) with serine antisense (AS2), using normal cDNA (Lane 3) and tumor cDNA (Lane 4).

FIG. 4 shows amplification with cysteine protease redundant primers. Normal (Lane 1), low malignant potential (Lane 2), serious carcinoma (Lane 3), mucinous carcinoma (Lane 4), and clear cell carcinoma (Lane 5).

FIG. 5 shows amplification with metallo-protease redundant primers. Normal (Lane 1), low malignant potential (Lane 2), serious carcinoma (Lane 3), mucinous carcinoma (Lane 4), and clear cell carcinoma (Lane 5).

FIG. 6 shows amplification with specific primers directed towards the serine protease, hepsin. Expression in normal (Lanes 1-3), low malignant potential tumors (Lanes 4-8), and ovarian carcinomas (Lanes 9-12).

FIG. 7 shows hepsin expression levels in normal, low malignant potential tumors, and ovarian carcinomas. S=serious, M=mucinous, LMP=low malignant potential.

FIG. 8 shows serine protease stratum corneum chymotrypsin enzyme (SCCE) expression in normal, low malignant potential tumors, and ovarian carcinomas.

FIG. 9 shows metallo-protease PUMP-1 (MMP-7) gene expression in normal (lanes 1-2) and ovarian carcinomas tissue (Lanes 3-10).

FIG. 10A shows Northern blot analysis of hepsin expression in normal ovary and ovarian carcinomas. Lane 1, normal ovary (case 10); lane 2, serous carcinoma (case 35); lane 3, mucinous carcinoma (case 48); lane 4, endometrioid carcinoma (case 51); and lane 5, clear cell carcinoma (case 54). In cases 35, 51 and 54, more than a 10-fold increase in the hepsin 1.8 kb transcript abundance was observed. FIG. 10B shows Northern blot analysis of hepsin in normal human fetal. FIG. 10C shows Northern blot analysis of hepsin in adult tissues. Significant overexpression of the hepsin transcript is noted in both fetal liver and fetal kidney. Notably, hepsin overexpression is not observed in normal adult tissue. Slight expression above the background level is observed in the adult prostate.

FIG. 11A shows hepsin expression in normal (N), mucinous (M) and serous (S) low malignant potential (LMP) tumors and carcinomas (CA). β-tubulin was used as an internal control. FIG. 11B shows the ratio of hepsin:β-tubulin expression in normal ovary, LMP tumor, and ovarian carcinoma. Hepsin mRNA expression levels were significantly elevated in LMP tumors, (p<0.005) and carcinomas (p<0.0001) compared to levels in normal ovary. All 10 cases of normal ovaries showed a relatively low level of hepsin mRNA expression.

FIG. 12A shows northern blot analysis of mRNA expression of the SCCE gene in fetal tissue. FIG. 12B shows northern blot analysis of mRNA expression of the SCCE gene in ovarian tissue.

FIG. 13A shows a comparison of quantitative PCR of SCCE cDNA from normal ovary and ovarian carcinomas. FIG. 13B shows a bar graph comparing the ratio of SCCE to β-tubulin in 10 normal and 44 ovarian carcinoma tissues.

FIG. 14 shows a comparison by quantitative PCR of normal and ovarian carcinoma expression of mRNA for protease M.

FIG. 15 shows the TADG-12 catalytic domain including an insert near the His 5′-end.

FIG. 16A shows northern blot analysis comparing TADG-14 expression in normal and ovarian carcinoma tissues. FIG. 16B shows preliminary quantitative PCR amplification of normal and carcinoma cDNAs using specific primers for TADG-14.

FIG. 17A shows northern blot analysis of the PUMP-1 gene in human fetal tissue. FIG. 17B shows northern blot analysis of the PUMP-1 gene in normal ovary and ovarian carcinomas.

FIG. 18A shows a comparison of PUMP-1 expression in normal and carcinoma tissues using quantitative PCR with an internal β-tubulin control. FIG. 18B shows the ratio of mRNA expression of PUMP-1 compared to the internal control β-tubulin in 10 normal and 44 ovarian carcinomas.

FIG. 19 shows a comparison of PCR amplified products for the hepsin, SCCE, protease M, PUMP-1 and Cathepsin L genes.

FIG. 20 shows CD8⁺ CTL recognition of hepsin 170-178 peptide in a 5 hr ⁵¹Cr release assay. Targets were LCL loaded with hepsin 170-178 (closed circles) and control LCL (open circles).

FIG. 21 shows CD8⁺ CTL recognition of hepsin 172-180 peptide in a 5 hr ⁵¹Cr release assay. Targets were LCL loaded with hepsin 172-180 (closed circles) and control LCL (open circles).

FIG. 22 shows CD8⁺ CTL recognition of hepsin 42-51 peptide in a 5 hr ⁵¹Cr release assay. Targets were LCL loaded with hepsin 42-51 (squares), control LCL (triangles) and K562 cells (diamonds).

FIG. 23 shows CD8⁺ CTL recognition of hepsin 284-293 peptide in a 5 hr ⁵¹Cr release assay. Targets were LCL loaded with hepsin 284-293 (closed circles) and control LCL (open circles).

FIG. 24 shows CD8⁺ CTL recognition of hepsin 308-317 peptide in a 5 hr ⁵¹Cr release assay. Targets cells were LCL loaded with or without hepsin 308-317.

FIG. 25 shows hepsin-specific CD4⁺ T cell and CD8⁺ T cell proliferative responses stimulated by full length hepsin protein. Solid histograms represent the stimulation index for T cells stimulated with hepsin-loaded dendritic cells, and open histograms represent the stimulation index for T cells stimulated with control dendritic cells.

### DETAILED DESCRIPTION OF THE INVENTION

This invention identifies hepsin protease as a marker for ovarian tumor cells. In various combinations with other proteases, hepsin expression is characteristic of individual tumor types. Such information can provide the basis for diagnostic tests (assays or immunohistochemistry) and prognostic evaluation, depending on the display pattern. Long-term treatment of tumor growth, invasion and metastasis has not succeeded with existing chemotherapeutic agents. Most tumors become resistant to drugs after multiple cycles of chemotherapy. The present invention identifies hepsin as a new therapeutic intervention target utilizing either antibodies directed at the protease, antisense vehicles for downregulation or protease inhibitors for the design of new drugs.

A primary object of the present invention is a method for detecting the presence of malignant hyperplasia in a tissue sample. The cancer is detected by analyzing a biological sample for the presence of markers to proteases that are specific indicators of certain types of cancer cells. This object may be accomplished by isolating mRNA from a sample or by detection of proteins by polyclonal or preferably monoclonal antibodies. When using mRNA detection, the method may be carried out by converting the isolated mRNA to cDNA according to standard methods; treating the converted cDNA with amplification reaction reagents, such as cDNA PCR reaction reagents, in a container along with an appropriate mixture of nucleic acid primers selected from the list in Table 2; reacting the contents of the container to produce amplification products; and analyzing the amplification products to detect the presence of malignant hyperplasia markers in the sample. The analyzing step may be accomplished using Northern Blot analysis to detect the presence of malignant hyperplasia markers in the amplification product. Northern Blot analysis is known in the art. The analysis step may be further accomplished by quantitatively detecting the presence of malignant hyperplasia marker in the amplification products, and comparing the quantity of marker detected against a panel of expected values for known presence or absence in normal and malignant tissue derived using similar primers.

The present invention also provides various nucleic acid sequences that are useful in the methods disclosed herein. These nucleic acid sequences are listed in Table 2. It is anticipated that these nucleic acid sequences be used in mixtures to accomplish the utility of this invention. The skilled artisan may be able to develop other nucleic acid sequences and mixtures thereof to accomplish the benefit of this invention, but it is advantageous to have the sequences listed in Table 2 available without undue experimentation.

The present invention provides a method for detecting malignant hyperplasia in a biological sample, comprising the steps of isolating mRNA from the sample; and detecting hepsin mRNA in the sample. The presence of the hepsin mRNA in the sample is indicative of the presence of malignant hyperplasia, wherein the absence of the hepsin mRNA in the sample is indicative of the absence of malignant hyperplasia. This method may further comprise the step of comparing the hepsin mRNA to reference information, wherein the comparison provides a diagnosis and/or determines a treatment of the malignant hyperplasia. A typical means of detection of hepsin mRNA is by PCR amplification, which, preferably, uses primers shown in SEQ ID NO: 8 and SEQ ID NO: 9. Representative biological samples are blood, urine, saliva, tears, interstitial fluid, ascites fluid, tumor tissue biopsy and circulating tumor cells.

The present invention is further directed toward a method of inhibiting expression of hepsin in a cell, comprising the step of introducing into a cell a vector comprises a hepsin gene operably linked in opposite orientation to elements necessary for expression, wherein expression of the vector produces hepsin antisense mRNA in the cell. The hepsin antisense mRNA hybridizes to endogenous hepsin mRNA, thereby inhibiting expression of hepsin in the cell.

The present invention is still further directed toward a method of inhibiting a hepsin protein in a cell, comprising the step of introducing an antibody into a cell, wherein the antibody is specific for a hepsin protein or a fragment thereof. Binding of the antibody to hepsin inhibits the hepsin protein. Preferably, the hepsin fragment is a 9-residue fragment up to a 20-residue fragment, and more preferably, the fragment is SEQ ID NOS: 28, 29, 30, 31, 88, 89, 108, 109, 128, 129, 148, 149, 150, 151, 152, 153, 154, 189, 190 or 191.

The present invention is also directed toward a method of targeted therapy to an individual, comprising the step of administering a compound to an individual, wherein the compound has a targeting moiety and a therapeutic moiety, and wherein the targeting moiety is specific for hepsin. Preferably, the targeting moiety is an antibody specific for hepsin or a ligand or ligand binding domain that binds hepsin. Likewise, the therapeutic moiety is preferably a radioisotope, a toxin, a chemotherapeutic agent, an immune stimulant or cytotoxic agent. Generally, the individual suffers from a disease such as ovarian cancer, lung cancer, prostate cancer, colon cancer or another cancer in which hepsin is overexpressed.

The present invention is additionally directed toward a method of vaccinating an individual against hepsin, comprising the steps of inoculating an individual with an expression vector encoding a hepsin protein or a fragment thereof. Expression of the hepsin protein, or fragment thereof, elicits an immune response in the individual, thereby vaccinating the individual against hepsin. Generally, this method is applicable when the individual has cancer or is at risk of getting a cancer such as ovarian cancer, lung cancer, prostate cancer and colon cancer. Sequences of preferred hepsin proteins or fragment thereof are shown in SEQ ID NOS: 28, 29, 30, 31, 88, 89, 108, 109, 128, 129, 148, 149, 150, 151, 152, 153, 154, 189, 190 and 191.

The present invention is yet directed toward a method of producing immune-activated cells directed toward hepsin, comprising the steps of exposing immune cells to hepsin protein or fragment thereof. Typically, exposure to hepsin protein or fragment thereof activates the immune cells, thereby producing immune-activated cells directed toward hepsin. Generally, the immune-activated cells are B-cells, T-cells and/or dendritic cells. Preferably, the hepsin fragment is a 9-residue fragment up to a 20-residue fragment, and more preferably, the fragment is SEQ ID NOS: 28, 29, 30, 31, 88, 89, 108, 109, 128, 129, 148, 149, 150, 151, 152, 153, 154, 189, 190 or 191. Oftentimes, the dendritic cells are isolated from an individual prior to exposure and then reintroduced into the individual subsequent to the exposure. Typically, the individual has cancer or is at risk of getting a cancer such as ovarian cancer, lung cancer, prostate cancer and colon cancer.

The present invention also provides methods of immunotherapy targeted toward hepsin in an individual. The methods involve generating dendritic cells in vitro from peripheral blood drawn from the individual, loading/introducing these dendritic cells with hepsin protein or a fragment thereof by lipofection or other means, then transferring these dendritic cells back to the individual in single or multiple doses. Hepsin may also be expressed in these dendritic cells following transduction with a recombinant DNA vector. Alternatively, hepsin-loaded or hepsin-expressing dendritic cells can be used to stimulate hepsin-specific T cell responses in vitro, followed by adoptive immunotherapy in which the individual is given autologous hepsin-specific T cells. Typically, the individual has cancer or is at risk of getting a cancer such as ovarian cancer, lung cancer, prostate cancer and colon cancer. In general, a full length or a fragment of hepsin protein is expressed in the isolated dendritic cells. Preferably, the fragment is a 9-residue fragment up to a 20-residue fragment, and more preferably, the fragment is SEQ D NOS: 28, 29, 30, 31, 88, 89, 108, 109, 128, 129, 148, 149, 150, 151, 152, 153, 154, 189, 190 or 191.

The present invention is further directed toward an immunogenic composition, comprising an appropriate adjuvant and an immunogenic full length hepsin protein or a fragment thereof. Preferably, the fragment is a 9-residue fragment up to a 20-residue fragment, and more preferably, the fragment is SEQ ID NOS: 28, 29, 30, 31, 88, 89, 108, 109, 128, 129, 148, 149, 150, 151, 152, 153, 154, 189, 190 or 191.

The present invention is further directed toward an oligonucleotide having a sequence complementary to SEQ ID NO: 188 or a fragment thereof. The present invention further provides a composition comprising the above-described oligonucleotide and a physiologically acceptable carrier, and a method of treating a neoplastic state in an individual in need of such treatment, comprising the step of administering to the individual an effective dose of the above-described oligonucleotide. Typically, the neoplastic state may be ovarian cancer, breast cancer, lung cancer, colon cancer, prostate cancer or another cancer in which hepsin is overexpressed.

The present invention is still further directed toward a method of screening for compounds that inhibit hepsin activity, comprising the steps of contacting a sample with a compound, wherein the sample comprises hepsin protein; and assaying for hepsin protease activity. A decrease in the hepsin protease activity in the presence of the compound relative to hepsin protease activity in the absence of the compound is indicative of a compound that inhibits hepsin activity.

It will be apparent to one skilled in the art that various substitutions and modifications may be made to the invention disclosed herein without departing from the scope and spirit of the invention.

In accordance with the present invention there may be employed conventional molecular biology, microbiology, and recombinant DNA techniques within the skill of the art. Such techniques are explained fully in the literature. See, e.g., Maniatis, Fritsch & Sambrook, “Molecular Cloning: A Laboratory Manual (1982); “DNA Cloning: A Practical Approach,” Volumes I and II (D. N. Glover ed. 1985); “Oligonucleotide Synthesis” (M. J. Gait ed. 1984); “Nucleic Acid Hybridization” (B. D. Hames & S. J. Higgins eds. 1985); “Transcription and Translation” (B. D. Hames & S. J. Higgins eds. 1984); “Animal Cell Culture” (R. I. Freshney, ed. 1986); “Immobilized Cells And Enzymes” (IRL Press, 1986); B. Perbal, “A Practical Guide To Molecular Cloning” (1984).

Therefore, if appearing herein, the following terms shall have the definitions set out below.

As used herein, the term “cDNA” shall refer to the DNA copy of the mRNA transcript of a gene.

As used herein, the term “PCR” refers to the polymerase chain reaction that is the subject of U.S. Pat. Nos. 4,683,195 and 4,683,202 to Mullis, as well as other improvements now known in the art.

The present invention comprises a vector comprising a DNA sequence which encodes a hepsin protein or a fragment thereof, wherein said vector is capable of replication in a host, and comprises, in operable linkage: a) an origin of replication; b) a promoter; and c) a DNA sequence coding for said hepsin protein. Preferably, the vector of the present invention contains a portion of the DNA sequence shown in SEQ ID NO: 188. Vectors may be used to amplify and/or express nucleic acid encoding a hepsin protein, a fragment of hepsin protein, or an antisense hepsin mRNA. Furthermore, the vectors may express nucleic acid encoding a fusion protein comprising an immunologically active component and a hepsin protein or a fragment thereof. These vectors would be useful in methods of vaccination against hepsin in an individual.

An expression vector is a replicable construct in which a nucleic acid sequence encoding a polypeptide is operably linked to suitable control sequences capable of effecting expression of the polypeptide in a cell. The need for such control sequences will vary depending upon the cell selected and the transformation method chosen. Generally, control sequences include a transcriptional promoter and/or enhancer, suitable mRNA ribosomal binding sites and sequences which control the termination of transcription and translation. Methods that are well known to those skilled in the art can be used to construct expression vectors containing appropriate transcriptional and translational control signals. See, for example, techniques described in Sambrook et al., 1989, Molecular Cloning: A Laboratory Manual (2nd Ed.), Cold Spring Harbor Press, N.Y. A gene and its transcription control sequences are defined as being “operably linked” if the transcription control sequences effectively control transcription of the gene. Vectors of the invention include, but are not limited to, plasmid vectors and viral vectors. Preferred viral vectors of the invention are those derived from retroviruses, adenovirus, adeno-associated virus, SV40 virus, or herpes viruses.

As used herein, the term “host” is meant to include not only prokaryotes but also eukaryotes such as yeast, plant and animal cells. A recombinant DNA molecule or gene which encodes a human hepsin protein of the present invention can be used to transform a host using any of the techniques commonly known to those of ordinary skill in the art. Especially preferred is the use of a vector containing coding sequences for the gene that encodes a human hepsin protein of the present invention for purposes of prokaryote transformation. Prokaryotic hosts may include E. coli, S. tymphimurium, Serratia marcescens and Bacillus subtilis. Eukaryotic hosts include yeasts such as Pichia pastoris, mammalian cells and insect cells.

The term “oligonucleotide”, as used herein, is defined as a molecule comprised of two or more ribonucleotides, preferably more than three. Its exact size will depend upon many factors, which, in turn, depend upon the ultimate function and use of the oligonucleotide. The term “primer”, as used herein, refers to an oligonucleotide, whether occurring naturally, as in a purified restriction digest, or produced synthetically, and which is capable of initiating synthesis of a strand complementary to a nucleic acid when placed under appropriate conditions, i.e., in the presence of nucleotides and an inducing agent, such as a DNA polymerase, and at a suitable temperature and pH. The primer may be either single-stranded or double-stranded and must be sufficiently long to prime the synthesis of the desired extension product in the presence of the inducing agent. The exact length of the primer will depend upon many factors, including temperature, sequence and/or homology of primer and the method used. For example, in diagnostic applications, the oligonucleotide primer typically contains 15-25 or more nucleotides, depending upon the complexity of the target sequence, although it may contain fewer nucleotides.

The primers herein are selected to be “substantially” complementary to particular target DNA sequences. This means that the primers must be sufficiently complementary to hybridize with their respective strands. Therefore, the primer sequence need not reflect the exact sequence of the template. For example, a non-complementary nucleotide fragment, i.e., containing a restriction site, may be attached to the 5′ end of the primer, with the remainder of the primer sequence being complementary to the strand. Alternatively, non-complementary bases or longer sequences can be interspersed into the primer, provided that the primer sequence has sufficient complementary with the sequence to hybridize therewith and form the template for synthesis of the extension product.

The probe to which the DNA of the invention hybridizes preferably consists of a sequence of at least 20 consecutive nucleotides, more preferably 40 nucleotides, even more preferably 50 nucleotides, and most preferably 100 nucleotides or more (up to 100%) of the coding sequence of the nucleotides listed in SEQ ID NO: 188 or the complement thereof. Such a probe is useful for detecting expression of hepsin in a cell by a method including the steps of (a) contacting mRNA obtained from the cell with a labeled hepsin hybridization probe; and (b) detecting hybridization of the probe with the mRNA.

As used herein, “substantially pure DNA” means DNA that is not part of a milieu in which the DNA naturally occurs, by virtue of separation (partial or total purification) of some or all of the molecules of that milieu, or by virtue of alteration of sequences that flank the claimed DNA. The term therefore includes, for example, a recombinant DNA which is incorporated into a vector, into an autonomously replicating plasmid or virus, or into the genomic DNA of a prokaryote or eukaryote; or which exists as a separate molecule, e.g., a cDNA or a genomic or cDNA fragment produced by polymerase chain reaction (PCR) or restriction endonuclease digestion) independent of other sequences. It also includes a recombinant DNA which is part of a hybrid gene encoding additional polypeptide sequence, e.g., a fusion protein. Also included is a recombinant DNA which includes a portion of the nucleotides listed in SEQ D NO: 188 and which encodes an alternative splice variant of hepsin.

The DNA may have at least about 70% sequence identity to the coding sequence of the nucleotides listed in SEQ ID NO: 188, preferably at least 75% (e.g., at least 80%); and most preferably at least 90%. The identity between two sequences is a direct function of the number of matching or identical positions. When a position in both of the two sequences is occupied by the same monomeric subunit, e.g., if a given position is occupied by an adenine in each of two DNA molecules, then they are identical at that position. For example, if 7 positions in a sequence 10 nucleotides in length are identical to the corresponding positions in a second 10-nucleotide sequence, then the two sequences have 70% sequence identity. The length of comparison sequences will generally be at least 50 nucleotides, preferably at least 60 nucleotides, more preferably at least 75 nucleotides, and most preferably 100 nucleotides. Sequence identity is typically measured using sequence analysis software (e.g., Sequence Analysis Software Package of the Genetics Computer Group (GCG), University of Wisconsin Biotechnology Center, 1710 University Avenue, Madison, Wis. 53705).

Further included in this invention are hepsin proteins which are encoded, at least in part, by portions of SEQ ID NO: 188, e.g., products of alternative mRNA splicing or alternative protein processing events, or in which a section of hepsin sequence has been deleted. The fragment, or the intact hepsin polypeptide, may be covalently linked to another polypeptide, e.g., one which acts as a label, a ligand or a means to increase antigenicity.

A substantially pure hepsin protein may be obtained, for example, by extraction from a natural source; by expression of a recombinant nucleic acid encoding a hepsin polypeptide; or by chemically synthesizing the protein. Purity can be measured by any appropriate method, e.g., column chromatography, such as immunoaffinity chromatography using an antibody specific for hepsin, polyacrylamide gel electrophoresis, or HPLC analysis. A protein is substantially free of naturally associated components when it is separated from at least some of those contaminants that accompany it in its natural state. Thus, a protein which is chemically synthesized or produced in a cellular system different from the cell from which it naturally originates will be, by definition, substantially free from its naturally associated components. Accordingly, substantially pure proteins include eukaryotic proteins synthesized in E. coli, other prokaryotes, or any other organism in which they do not naturally occur.

In addition to substantially full-length proteins, the invention also includes fragments (e.g., antigenic fragments) of the hepsin protein. As used herein, “fragment,” as applied to a polypeptide, will ordinarily be at least 10 residues, more typically at least 20 residues, and preferably at least 30, e.g., 50, residues in length, but less than the entire, intact sequence. Fragments of the hepsin protein can be generated by methods known to those skilled in the art, e.g., by enzymatic digestion of naturally occurring or recombinant hepsin protein, by recombinant DNA techniques using an expression vector that encodes a defined fragment of hepsin, or by chemical synthesis. The ability of a candidate fragment to exhibit a characteristic of hepsin, e.g., binding to an antibody specific for hepsin, can be assessed by methods known in the art. Purified hepsin or antigenic fragments of hepsin can be used to generate new antibodies or to test existing antibodies, e.g., as positive controls in a diagnostic assay, by employing standard protocols known to those skilled in the art. Included in this invention is polyclonal antisera generated by using hepsin or a fragment of hepsin as the immunogen in, e.g., rabbits. Standard protocols for monoclonal and polyclonal antibody production known to those skilled in this art are employed. The monoclonal antibodies generated by this procedure can be screened for the ability to identify recombinant hepsin cDNA clones, and to distinguish them from other cDNA clones.

The invention encompasses not only an intact anti-hepsin monoclonal antibody, but also an immunologically-active antibody fragment, e.g., a Fab or (Fab)₂ fragment; an engineered single chain Fv molecule; or a chimeric molecule, e.g., an antibody which contains the binding specificity of one antibody, e.g., of murine origin, and the remaining portions of another antibody, e.g., of human origin.

In one embodiment, the antibody, or a fragment thereof, may be linked to a toxin or to a detectable label, e.g., a radioactive label, non-radioactive isotopic label, fluorescent label, chemiluminescent label, paramagnetic label, enzyme label, or colorimetric label. Examples of suitable toxins include diphtheria toxin, Pseudomonas exotoxin A, ricin, and cholera toxin. Examples of suitable enzyme labels include malate hydrogenase, staphylococcal nuclease, delta-5-steroid isomerase, alcohol dehydrogenase, alpha-glycerol phosphate dehydrogenase, triose phosphate isomerase, peroxidase, alkaline phosphatase, asparaginase, glucose oxidase, beta-galactosidase, ribonuclease, urease, catalase, glucose-6-phosphate dehydrogenase, glucoamylase, acetylcholinesterase, etc. Examples of suitable radioisotopic labels include ³H, ¹²⁵I, ¹³¹I , ³²P, ³⁵S, ¹⁴C, etc.

Paramagnetic isotopes for purposes of in vivo diagnosis can also be used according to the methods of this invention. There are numerous examples of elements that are useful in magnetic resonance imaging. For discussions on in vivo nuclear magnetic resonance imaging, see, for example, Schaefer et al., (1989) JACC 14, 472-480; Shreve et al., (1986) Magn. Reson. Med. 3, 336-340; Wolf, G. L., (1984) Physiol. Chem. Phys. Med. NMR 16, 93-95; Wesbey et al., (1984) Physiol. Chem. Phys. Med. NMR 16, 145-155; Runge et al., (1984) Invest. Radiol. 19, 408-415. Examples of suitable fluorescent labels include a fluorescein label, an isothiocyalate label, a rhodamine label, a phycoerythrin label, a phycocyanin label, an allophycocyanin label, an ophthaldehyde label, a fluorescamine label, etc. Examples of chemiluminescent labels include a luminal label, an isoluminal label, an aromatic acridinium ester label, an imidazole label, an acridinium salt label, an oxalate ester label, a luciferin label, a luciferase label, an aequorin label, etc.

Those of ordinary skill in the art will know of other suitable labels which may be employed in accordance with the present invention. The binding of these labels to antibodies or fragments thereof can be accomplished using standard techniques commonly known and used by those of ordinary skill in the art. Typical techniques are described by Kennedy et al., (1976) Clin. Chim. Acta 70, 1-31; and Schurs et al., (1977) Clin. Chim. Acta 81, 1-40. Coupling techniques mentioned in the latter are the glutaraldehyde method, the periodate method, the dimaleimide method, the m-maleimidobenzyl-N-hydroxy-succinimide ester method. All of these methods are incorporated by reference herein.

Also within the invention is a method of detecting hepsin protein in a biological sample, which includes the steps of contacting the sample with the labeled antibody, e.g., radioactively tagged antibody specific for hepsin, and determining whether the antibody binds to a component of the sample. Antibodies to the hepsin protein can be used in an immunoassay to detect increased levels of hepsin protein expression in tissues suspected of neoplastic transformation. These same uses can be achieved with Northern blot assays and analyses.

As described herein, the invention provides a number of diagnostic advantages and uses. For example, the hepsin protein is useful in diagnosing cancer in different tissues since this protein is highly overexpressed in tumor cells. Antibodies (or antigen-binding fragments thereof) which bind to an epitope specific for hepsin are useful in a method of detecting hepsin protein in a biological sample for diagnosis of cancerous or neoplastic transformation. This method includes the steps of obtaining a biological sample (e.g., cells, blood, plasma, tissue, etc.) from a patient suspected of having cancer, contacting the sample with a labeled antibody, e.g., radioactively tagged antibody, specific for hepsin, and detecting the hepsin protein using standard immunoassay techniques such as an ELISA. Antibody binding to the biological sample indicates that the sample contains a component which specifically binds to an epitope within hepsin.

Likewise, a standard Northern blot assay can be used to ascertain the relative amounts of hepsin mRNA in a cell or tissue obtained from a patient suspected of having cancer, in accordance with conventional Northern hybridization techniques known to those of ordinary skill in the art. This Northern assay uses a hybridization probe, e.g., radiolabelled hepsin cDNA, either containing the full-length, single stranded DNA having a sequence complementary to SEQ ID NO: 188, or a fragment of that DNA sequence at least 20, preferably at least 30, more preferably at least 50, and most preferably at least 100 consecutive nucleotides in length. The DNA hybridization probe can be labeled by any of the many different methods known to those skilled in this art.

The following examples are given for the purpose of illustrating various embodiments of the invention and are not meant to limit the present invention in any fashion:

### EXAMPLE 1

### Amplification of Serine Proteases Using Redundant and Specific Primers

Only cDNA preparations deemed free of genomic DNA were used for gene expression analysis. Redundant primers were prepared for serine proteases, metallo-proteases and cysteine protease. The primers were synthesized to consensus sequences of amino acid surrounding the catalytic triad for serine proteases, viz. histidine . . . aspartate . . . and serine. The sequences of both sense (histidine & aspartate) and antisense (aspartate and serine) redundant primers are shown in Table 2.

Several protease entities were identified and subcloned from PCR amplification of cDNA derived from serous cystadenocarcinomas. Therefore, the proteases described herein are reflective of surface activities for this type of carcinoma, the most common form of ovarian cancer. Applicant also shows PCR amplification bands of similar base pair size unique to the mucinous tumor type and the clear cell type. About 20-25% of ovarian cancers are classified as either mucinous, clear cell, or endometrioid.

To determine the identity of the PCR products, all the appropriate bands were ligated into Promega T-vector plasmid and the ligation product was used to transform JM109 cells (Promega) grown on selective media. After selection and culturing of individual colonies, plasmid DNA was isolated by means of the WIZARD MINIPREP™ DNA purification system (Promega). Inserts were sequenced using a Prism Ready Reaction Dydeoxy Terminators cycle sequencing kit (Applied Biosystems). Residual dye terminators were removed from the completed sequencing reaction using a CENTRISEP SPIN™ column (Princeton Separation). Samples were loaded into an Applied Biosystems Model 373A DNA sequencing system. The results of subcloning and sequencing for the serine protease primers are shown in Table 3.

TABLE 2

| | | SEQ |
|---|---|---|
| PCR Primers | 5′→3′ | ID NO: |
| Redundant Primers: | | |
| Serine Protease (histidine) = S1 | tgggtigtiacigcigcica(ct)tg | 1 |
| Serine Protease (aspartic acid) = AS1 | a(ag)ia(ag)igciatitcitticc | 2 |
| Serine Protease (serine) = AS11 | a(ag)iggiccicci(cg)(ta)(ag)tcicc | 3 |
| Cysteine Protease - sense | ca(ag)ggica(ag)tg(ct)ggi(ta)(cg)itg(ct) | 4 |
| | tgg | |
| Cysteine Protease - antisense | taiccicc(ag)tt(ag)caicc(ct)tc | 5 |
| Metallo Protease - sense | cci(ac)gitg(tc)ggi(ga)(ta)icciga | 6 |
| Metallo Protease - antisense | tt(ag)tgicciai(ct)tc(ag)tg | 7 |
| Specific Primers: | | |
| Serine Protease (hepsin) = sense | tgtcccgatggcgagtgttt | 8 |
| Serine Protease (hepsin) = antisense | cctgttggccatagtactgc | 9 |
| Serine Protease (SCCE) = sense | agatgaatgagtacaccgtg | 10 |
| Serine Protease (SCCE) = antisense | ccagtaagtccttgtaaacc | 11 |
| Serine Protease (Comp B) = sense | aagggacacgagagctgtat | 12 |
| Serine Protease (Comp B) = antisense | aagtggtagttggaggaagc | 13 |
| Serine Protease (Protease M) = sense | ctgtgatccaccctgactat | 20 |
| Serine Protease (Protease M) = antisense | caggtggatgtatgcacact | 21 |
| Serine Protease (TADG12) = sense (Ser10-s) | gcgcactgtgtttatgagat | 22 |
| Serine Protease (TADG12) = antisense (Ser10-as) | ctctttggcttgtacttgct | 23 |
| Serine Protease (TADG13) = sense | tgagggacatcattatgcac | 24 |
| Serine Protease (TADG13) = antisense | caagttttccccataattgg | 25 |
| Serine Protease (TADG14) = sense | acagtacgcctgggagacca | 26 |
| Serine Protease (TADG14) = antisense | ctgagacggtgcaattctgg | 27 |
| Cysteine Protease (Cath-L) = sense | attggagagagaaaggctac | 14 |
| Cysteine Protease (Cath-L) = antisense | cttgggattgtacttacagg | 15 |
| Metallo Protease (PUMP1) = sense | cttccaaagtggtcacctac | 16 |
| Metallo Protease (PUMP1) = antisense | ctagactgctaccatccgtc | 17 |

TABLE 3
Serine protease candidates

| Subclone | Primer Set | Gene Candidate |
|---|---|---|
| 1 | His-Ser | Hepsin |
| 2 | His-Ser | SCCE |
| 3 | His-Ser | Compliment B |
| 4 | His-Asp | Cofactor 1 |
| 5 | His-Asp | TADG-12* |
| 6 | His-Ser | TADG-13* |
| 7 | His-Ser | TADG-14* |
| 8 | His-Ser | Protease M |
| 9 | His-Ser | TADG-15* |

*indicates novel proteases

Sequencing of the PCR products derived from tumor cDNA confirms the potential candidacy of these genes. The three novel genes all have conserved residues within the catalytic triad sequence consistent with their membership in the serine protease family.

Applicant compared the PCR products amplified from normal and carcinoma cDNAs using sense-histidine and antisense-aspartate as well as sense-histidine and antisense-serine. The anticipated PCR products of approximately 200 bp and 500 bp for those pairs of primers were observed (aspartate is approximately 50-70 amino acids downstream from histidine, and serine is about 100-150 amino acids toward the carboxy end from histidine).

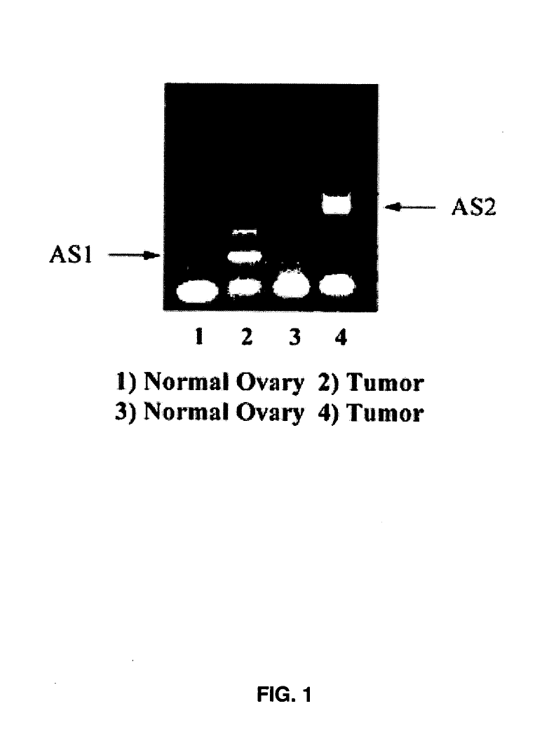

FIG. 1 shows a comparison of PCR products derived from normal and carcinoma cDNA as shown by staining in an agarose gel. Two distinct bands in Lane 2 were present in the primer pair sense-His/antisense ASP (AS1) and multiple bands of about 500 bp are noted in the carcinoma lane for the sense-His/antisense-Ser (AS2) primer pairs in Lane 4.

### EXAMPLE 2

### Northern Blots Analysis

Significant information can be obtained by examining the expression of these candidate genes by Northern blot. Analysis of normal adult multi-tissue blots offers the opportunity to identify normal tissues which may express the protease. Ultimately, if strategies for inhibition of proteases for therapeutic intervention are to be developed, it is essential to appreciate the expression of these genes in normal tissues.

Significant information is expected from Northern blot analysis of fetal tissue. Genes overexpressed in carcinomas are often highly expressed in organogenesis. As indicated, the hepsin gene cloned from hepatoma cells and overexpressed in ovarian carcinoma is overtly expressed in fetal liver. Hepsin gene expression was also detected in fetal kidney, and therefore, could be a candidate for expression in renal carcinomas.

Northern panels for examining expression of genes in a multi-tissue normal adult as well as fetal tissue are commercially available (CLONTECH). Such evaluation tools are not only important to confirm the overexpression of individual transcripts in tumor versus normal tissues, but also provides the opportunity to confirm transcript size, and to determine if alternate splicing or other transcript alteration may occur in ovarian carcinoma.

Northern blot analysis was performed as follows: 10 μg of mRNA was loaded onto a 1% formaldehyde-agarose gel, electrophoresed and blotted onto a HyBond-N⁺™ nylon membrane (Amersham). ³²P-labeled cDNA probes were made using Prime-a-Gene Labeling System™ (Promega). The PCR products amplified by specific primers were used as probes. Blots were prehybridized for 30 min and then hybridized for 60 min at 68° C. with ³²P-labeled cDNA probe in ExpressHyb™ Hybridization Solution (CLONTECH). Control hybridization to determine relative gel loading was accomplished using the β-tubulin probe.

Normal human tissues including spleen, thymus, prostate, testis, ovary, small intestine, colon, peripheral blood leukocyte, heart, brain, placenta, lung, liver, skeletal muscle, kidney, pancreas and normal human fetal tissues (Human Multiple Tissue Northern Blot; CLONTECH) were all examined using the same hybridization procedure.

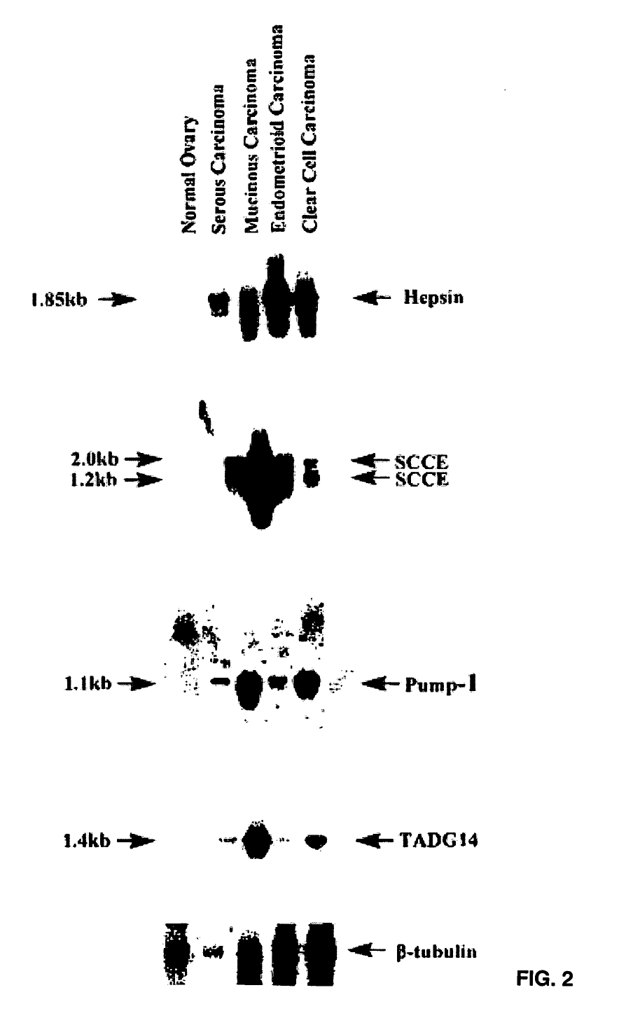

Experiments comparing PCR amplification in normal ovary and ovarian carcinoma suggested overexpression and/or alteration in mRNA transcript in tumor tissues. Northern blot analysis of TADG-14 confirms a transcript size of 1.4 kb and data indicate overexpression in ovarian carcinoma (FIG. 2). Isolation and purification using both PCR and a specific 250 bp PCR product to screen positive plaques yielded a 1.2 kb clone of TADG-14. Other proteases were amplified by the same method using the appropriate primers from Table 2.

### EXAMPLE 3

### PCR Products Corresponding to Serine, Cysteine and Metallo-Proteases

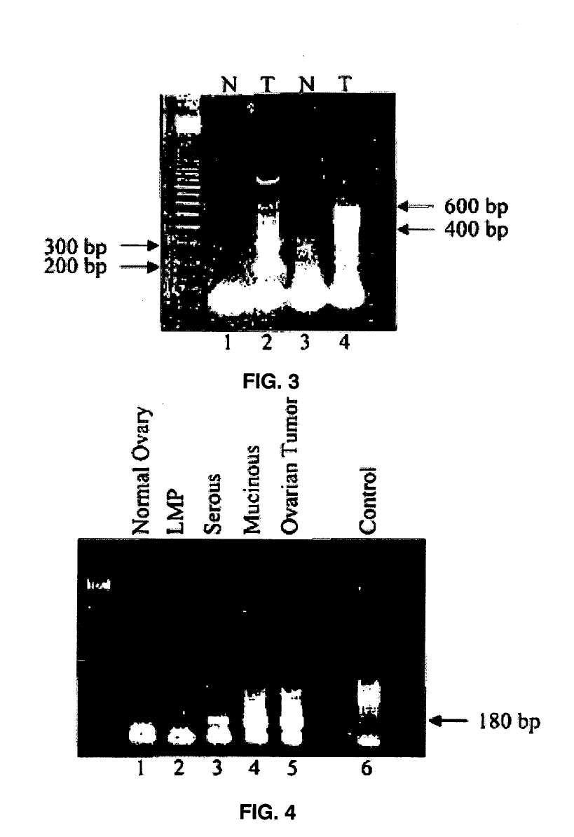

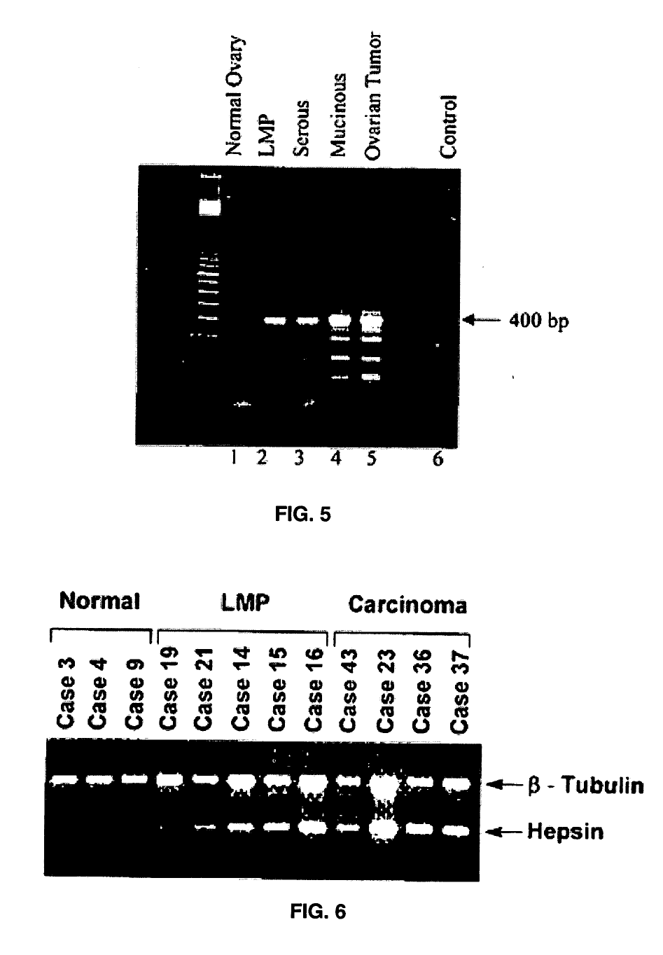

Based on their unique expression in either low malignant potential tumors or carcinomas, PCR-amplified cDNA products were cloned and sequenced and the appropriate gene identified based upon nucleotide and amino acid sequences stored in the GCG and EST databases. FIGS. 3, 4 & 5 show the PCR product displays comparing normal and carcinomatous tissues using redundant primers for serine proteases (FIG. 3), for cysteine proteases (FIG. 4) and for metallo-proteases (FIG. 5). Note the differential expression in the carcinoma tissues versus the normal tissues. The proteases were identified using redundant cDNA primers (see Table 2) directed towards conserved sequences that are associated with intrinsic enzyme activity (for serine proteases, cysteine proteases and metallo-proteases) by comparing mRNA expression in normal, low malignant potential and overt ovarian carcinoma tissues according to Sakanari et al. [Biochemistry 86, 4863-4867 (1989)].

### EXAMPLE 4

### Serine Proteases

For the serine protease group, using the histidine domain primer sense, S1, in combination with antisense primer AS2, the following proteases were identified:

(a) Hepsin, a trypsin-like serine protease cloned from hepatoma cells shown to be a cell surface protease essential for the growth of hepatoma cells in culture and highly expressed in hepatoma tumor cells (FIG. 3, Lane 4);

(b) Complement factor B protease (human factor IX), a protease involved in the coagulation cascade and associated with the production and accumulation of fibrin split products associated with tumor cells (FIG. 3, Lane 4). Compliment factor B belongs in the family of coagulation factors X (Christmas factor). As part of the intrinsic pathway, compliment factor B catalyzes the proteolytic activation of coagulation factor X in the presence of Ca²⁺ phospholipid and factor Villa e5; and

(c) A stratum corneum chymotryptic enzyme (SCCE) serine protease involved in desquarnation of skin cells from the human stratum corneum (FIG. 3, Lane 4). SCCE is expressed in keratinocytes of the epidermis and functions to degrade the cohesive structures in the cornified layer to allow continuous skin surface shedding.

### EXAMPLE 5

### Cysteine Proteases

In the cysteine protease group, using redundant sense and anti-sense primers for cysteine proteases, one unique PCR product was identified by overexpression in ovarian carcinoma when compared to normal ovarian tissue (FIG. 4, Lanes 3-5). Cloning and sequencing this PCR product identified a sequence of Cathepsin L, which is a lysomal cysteine protease whose expression and secretion is induced by malignant transformation, growth factors and tumor promoters. Many human tumors (including ovarian) express high levels of Cathepsin L. Cathepsin L cysteine protease belongs in the stromolysin family and has potent elastase and collagenase activities. Published data indicates increased levels in the serum of patients with mucinous cystadenocarcinoma of the ovary. It has not heretofore been shown to be expressed in other ovarian tumors.

### EXAMPLE 6

### Metallo-Proteases

Using redundant sense and anti-sense primers for the metallo-protease group, one unique PCR product was detected in the tumor tissue which was absent in normal ovarian tissue (FIG. 5, Lanes 2-5). Subcloning and sequencing this product indicates it has complete homology in the appropriate region with the so-called PUMP-1 (MMP-7) gene. This zinc-binding metallo-protease is expressed as a proenzyme with a signal sequence and is active in gelatin and collagenase digestion. PUMP-1 has also been shown to be induced and overexpressed in 9 of 10 colorectal carcinomas compared to normal colon tissue, suggesting a role for this substrate in the progression of this disease.

### EXAMPLE 7

### Expression of Hepsin

The mRNA overexpression of hepsin was detected and determined using quantitative PCR. Quantitative PCR was performed generally according to the method of Noonan et al. (3). The following oligonucleotide primers were used: hepsin forward 5′-TGTCCCGATGGCGAGTGTTT-3′ (SEQ ID NO: 8), and hepsin reverse 5′-CCTGTTGGCCATAGTA CTGC-3′ (SEQ ID NO: 9); β-tubulin forward 5′-TGCATTGACAACGAGGC-3′ (SEQ ID NO: 18), and β-tubulin reverse 5′-CTGTCTTGACATTGTTG-3′ (SEQ ID NO: 19).

β-tubulin was utilized as an internal control. The predicted sizes of the amplified genes were 282 bp for hepsin and 454 bp for β-tubulin. The primer sequences used in this study were designed according to the cDNA sequences described by Leytus et al. (4) for hepsin, and Hall et al. (5) for β-tubulin.

The PCR reaction mixture consisted of cDNA derived from 50 ng of mRNA converted by conventional techniques, 5 μmol of sense and antisense primers for both the hepsin gene and the β-tubulin gene, 200 μmol of dNTPs, 5 μCi of α-³²PdCTP and 0.25 units of Taq DNA polymerase with reaction buffer (Promega) in a final volume of 25 μl. The target sequences were amplified in parallel with the β-tubulin gene. Thirty cycles of PCR were carried out in a Thermal Cycler (Perkin-Elmer Cetus). Each cycle of PCR included 30 sec of denaturation at 95° C., 30 sec of annealing at 63° C. and 30 sec of extension at 72° C. The PCR products were separated on 2% agarose gels and the radioactivity of each PCR product was determined by using a Phosphorlmager™ (Molecular Dynamics). Student's t test was used for comparison of mean values.

Hepsin is a trypsin-like serine protease cloned from hepatoma cells. Hepsin is an extracellular protease (the enzyme includes a secretion signal sequence) which is anchored in the plasma membrane by its amino terminal domain, thereby exposing its catalytic domain to the extracellular matrix. Hepsin has also been shown to be expressed in breast cancer cell lines and peripheral nerve cells. Hepsin has never before been associated with ovarian carcinoma. Specific primers for the hepsin gene were synthesized and the expression of hepsin examined using Northern blots of fetal tissue and ovarian tissue (both normal and ovarian carcinoma).

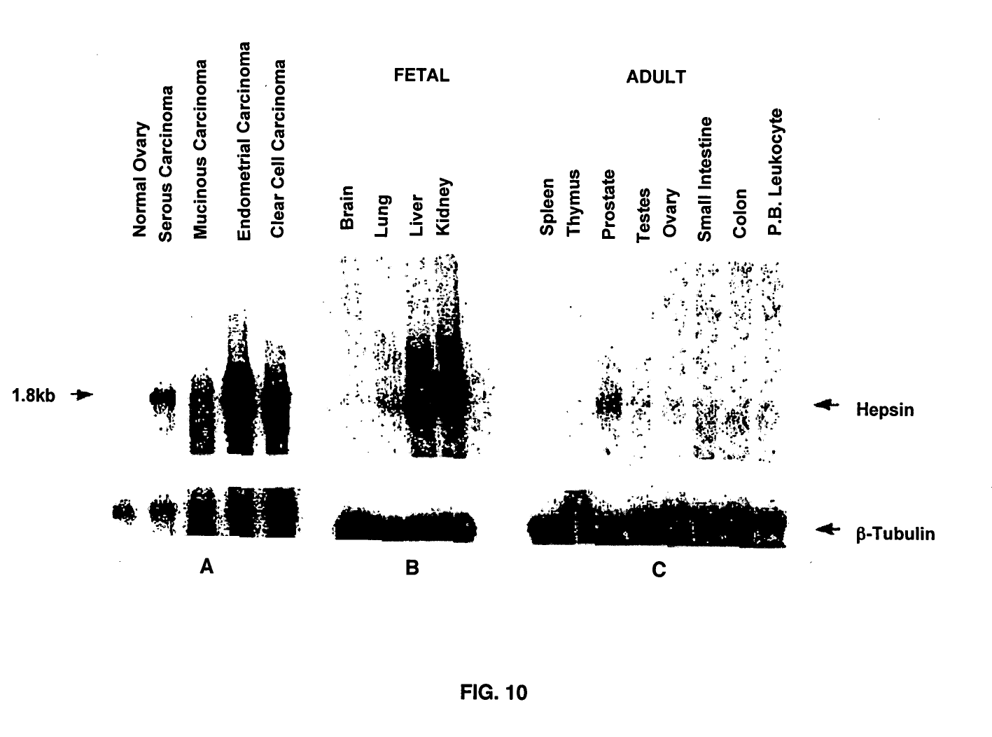

FIG. 10A shows that hepsin was expressed in ovarian carcinomas of different histologic types, but not in normal ovary. FIG. 10B shows that hepsin was expressed in fetal liver and fetal kidney as anticipated, but at very low levels or not at all in fetal brain and lung. FIG. 10C shows that hepsin overexpression is not observed in normal adult tissue. Slight expression above the background level is observed in the adult prostate. The mRNA identified in both Northern blots was the appropriate size for the hepsin transcript. The expression of hepsin was examined in 10 normal ovaries and 44 ovarian tumors using specific primers to β-tubulin and hepsin in a quantitative PCR assay, and found it to be linear over 35 cycles. Expression is presented as the ratio of ³²P-hepsin band to the internal control, the ³²P-β-tubulin band.

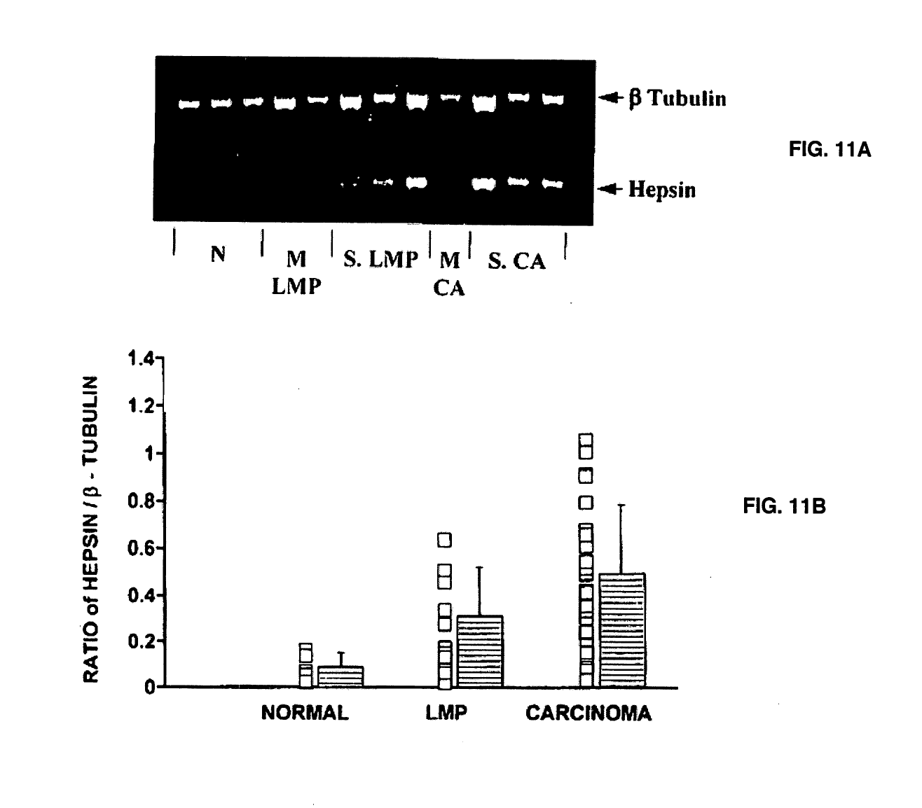

Hepsin expression was investigated in normal (N), mucinous (M) and serous (S) low malignant potential (LMP) tumors and carcinomas (CA). FIG. 11A shows quantitative PCR of hepsin and internal control β-tubulin. FIG. 11B shows the ratio of hepsin:β-tubulin expression in normal ovary, LMP tumor, and ovarian carcinoma. It was observed that Hepsin mRNA expression levels were significantly elevated in LMP tumors, (p<0.005) and carcinomas (p<0.0001) compared to levels in normal ovary. All 10 cases of normal ovaries showed a relatively low level of hepsin mRNA expression.

Hepsin mRNA is highly overexpressed in most histopathologic types of ovarian carcinomas including some low malignant potential tumors (FIGS. 11A-11B). Most noticeably, hepsin is highly expressed in serous, endometrioid and clear cell tumors tested. It is highly expressed in some mucinous tumors, but it is not overexpressed in the majority of such tumors.

A tumor tissue bank of fresh frozen tissue of ovarian carcinomas as shown in Table 4 was used for evaluation. Approximately 100 normal ovaries removed for medical reasons other than malignancy were obtained from surgery and were available as controls. From the tumor bank, approximately 100 carcinomas were evaluated encompassing most histological sub-types of ovarian carcinoma, including borderline or low-malignant potential tumors and overt carcinomas. The approach included using mRNA prepared from fresh frozen tissue (both normal and malignant) to compare expression of genes in normal, low malignant potential tumors and overt carcinomas. The cDNA prepared from polyA⁺ mRNA was deemed to be genomic DNA-free by checking all preparations with primers that encompassed a known intron-exon splice site using both β-tubulin and p53 primers.

TABLE 4
Ovarian cancer tissue bank

| | Total | Stage I/11 | Stage III/IV | No Stage |
|---|---|---|---|---|
| Serous | | | | |
| Malignant | 166 | 15 | 140 | 8 |
| LMP | 16 | 9 | 7 | 0 |
| Benign | 12 | 0 | 0 | 12 |
| Mucinous | | | | |
| Malignant | 26 | 6 | 14 | 6 |
| LMP | 28 | 25 | 3 | 0 |
| Benign | 3 | 0 | 0 | 3 |
| Endometrioid | | | | |
| Malignant | 38 | 17 | 21 | 0 |
| LMP | 2 | 2 | 0 | 0 |
| Benign | 0 | 0 | 0 | 0 |
| Other* | | | | |
| Malignant | 61 | 23 | 29 | 9 |
| LMP | 0 | 0 | 0 | 0 |
| Benign | 5 | 0 | 0 | 5 |

*Other category includes the following tumor types: Brenner's tumor, thecoma, teratoma, fibrothécoma, fibroma, granulosa cell, clear cell, germ cell, mixed mullerian, stromal, undifferentiated, and dysgerminoma.

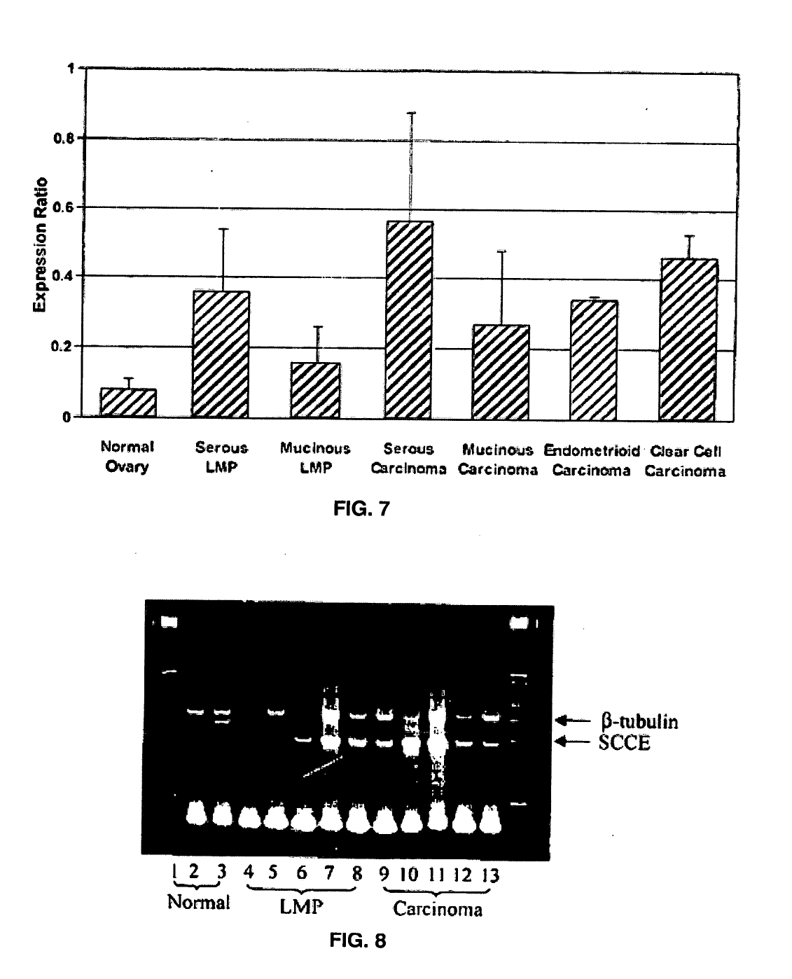

The expression of the serine protease hepsin gene in 8 normal, 11 low malignant potential tumors, and 14 carcinoma (both mucinous and serous type) by quantitative PCR using hepsin-specific primers (see Table 2) was determined (primers directed toward the β-tubulin message were used as an internal standard) (Table 5). These data confirm the overexpression of the hepsin surface protease gene in ovarian carcinoma, including both low malignant potential tumors and overt carcinoma. Expression of hepsin is increased over normal levels in low malignant potential tumors, and high stage tumors (Stage III) of this group have higher expression of hepsin when compared to low stage tumors (Stage 1) (Table 6). In overt carcinoma, serous tumors exhibit the highest levels of hepsin expression, while mucinous tumors express levels of hepsin comparable with the high stage low malignant potential group (FIGS. 6-7).

TABLE 5
Patient Characteristics and Expression of Hepsin Gene

| | | | | mRNA |
|---|---|---|---|---|
| | | | | expression |
| Case | Histological type^{a} | Stage/Grade | LN^{b} | of hepsin^{c} |
| 1 | normal ovary | | | n |
| 2 | normal ovary | | | n |
| 3 | normal ovary | | | n |
| 4 | normal ovary | | | n |
| 5 | normal ovary | | | n |
| 6 | normal ovary | | | n |
| 7 | normal ovary | | | n |
| 8 | normal ovary | | | n |
| 9 | normal ovary | | | n |
| 10 | normal ovary | | | n |
| 11 | S adenoma (LMP) | 1/1 | N | 4+ |
| 12 | S adenoma (LMP) | 1/1 | NE | 4+ |
| 13 | S adenoma (LMP) | 1/1 | NE | n |
| 14 | S adenoma (LMP) | 1/1 | N | 2+ |
| 15 | S adenoma (LMP) | 3/1 | P | 4+ |
| 16 | S adenoma (LMP) | 3/1 | P | 4+ |
| 17 | S adenoma (LMP) | 3/1 | P | 4+ |
| 18 | M adenoma (LMP) | 1/1 | NE | 4+ |
| 19 | M adenoma (LMP) | 1/1 | N | n |
| 20 | M adenoma (LMP) | 1/1 | N | n |
| 21 | M adenoma (LMP) | 1/1 | N | n |
| 22 | M adenoma (LMP) | 1/1 | NE | n |
| 23 | S carcinoma | 1/2 | N | 4+ |
| 24 | S carcinoma | 1/3 | N | 4+ |
| 25 | S carcinoma | 3/1 | NE | 2+ |
| 26 | S carcinoma | 3/2 | NE | 4+ |
| 27 | S carcinoma | 3/2 | P | 4+ |
| 28 | S carcinoma | 3/2 | NE | 2+ |
| 29 | S carcinoma | 3/3 | NE | 2+ |
| 30 | S carcinoma | 3/3 | NE | 4+ |
| 31 | S carcinoma | 3/3 | NE | 4+ |
| 32 | S carcinoma | 3/3 | NE | 4+ |
| 33 | S carcinoma | 3/3 | N | 4+ |
| 34 | S carcinoma | 3/3 | NE | n |
| 35 | S carcinoma | 3/3 | NE | 4+ |
| 36 | S carcinoma | 3/3 | NE | 4+ |
| 37 | S carcinoma | 3/3 | NE | 4+ |
| 38 | S carcinoma | 3/3 | N | 4+ |
| 39 | S carcinoma | 3/2 | NE | 2+ |
| 40 | S carcinoma | 3/3 | NE | 4+ |
| 41 | S carcinoma | 3/2 | NE | 4+ |
| 42 | M carcinoma | 1/2 | N | n |
| 43 | M carcinoma | 2/2 | NE | 4+ |
| 44 | M carcinoma | 2/2 | N | 4+ |
| 45 | M carcinoma | 3/1 | NE | n |
| 46 | M carcinoma | 3/2 | NE | 4+ |
| 47 | M carcinoma | 3/2 | NE | n |
| 48 | M carcinoma | 3/3 | NE | n |
| 49 | E carcinoma | 2/3 | N | 4+ |
| 50 | E carcinoma | 3/2 | NE | 4+ |
| 51 | E carcinoma | 3/3 | NE | 4+ |
| 52 | C carcinoma | 1/3 | N | 4+ |
| 53 | C carcinoma | 1/1 | N | 4+ |
| 54 | C carcinoma | 3/2 | P | 4+ |

^{a}S, serous; M, mucinous; E, endometrioid; C, clear cell;
^{b}LN, lymph node metastasis; P, positive; N, negative; NE, not examined;
^{c}n, normal range = mean ± 2 SD; 2+, mean ± 2 SD to ± 4 SD; 4+, mean ± 4 SD or greater.

TABLE 6
Overexpression of hepsin in normal ovaries and ovarian tumors

| | | | |
|---|---|---|---|
| | | Hepsin | Ratio of Hepsin |
| Type | N | Overexpression | to β-tubulin |

| | | | | |
|---|---|---|---|---|
| Normal | 10 | 0 | (0%) | 0.06 ± 0.05 |
| LMP | 12 | 7 | (58.3%) | 0.26 ± 0.19 |
| Serous | 7 | 6 | (85.7%) | 0.34 ± 0.20 |
| Mucinous | 5 | 1 | (20.0%) | 0.14 ± 0.12 |
| Carcinomous | 32 | 27 | (84.4%) | 0.46 ± 0.29 |
| Serous | 19 | 18 | (94.7%) | 0.56 ± 0.32 |
| Mucinous | 7 | 3 | (42.9%) | 0.26 ± 0.22 |
| Endometrioid | 3 | 3 | (100%) | 0.34 ± 0.01 |
| Clear Cell | 3 | 3 | (100%) | 0.45 ± 0.08 |

### EXAMPLE 8

### Expression of SCCE and PUMP-1

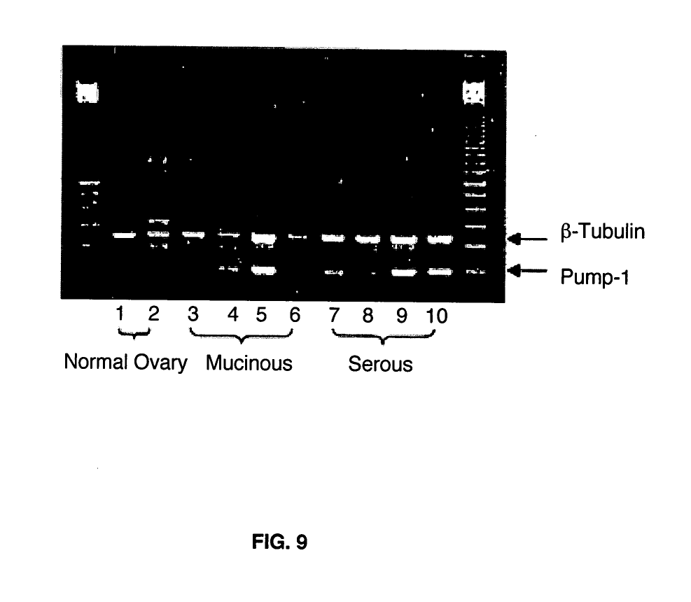

Studies using both SCCE-specific primers (FIG. 8) and PUMP-specific primers (FIG. 9) indicate overexpression of these proteases in ovarian carcinomas.

### EXAMPLE 9

### Summary of Proteases Detected Herein

Most of the proteases described herein were identified from the sense-His/antisense-Ser primer pair, yielding a 500 bp PCR product (FIG. 1, Lane 4). Some of the enzymes are familiar, a short summary of each follows.

### Stratum Corneum Chymotrypsin Enzyme (SCCE)

The PCR product identified was the catalytic domain of the sense-His/antisense-Ser of the stratum corneum chymotrypsin enzyme. This extracellular protease was cloned, sequenced and shown to be expressed on the surface of keratinocytes in the epidermis. Stratum corneum chymotrypsin enzyme is a chymotrypsin-like serine protease whose function is suggested to be in the catalytic degradation of intercellular cohesive structures in the stratum corneum layer of the skin. This degradation allows continuous shedding (desquamation) of cells from the skin surface. The subcellular localization of stratum corneum chymotrypsin enzyme is in the upper granular layer in the stratum corneum of normal non-palmoplantar skin and in the cohesive parts of hypertrophic plantar stratum corneum. Stratum corneum chymotrypsin enzyme is exclusively associated with the stratum corneum and has not so far been shown to be expressed in any carcinomatous tissues.

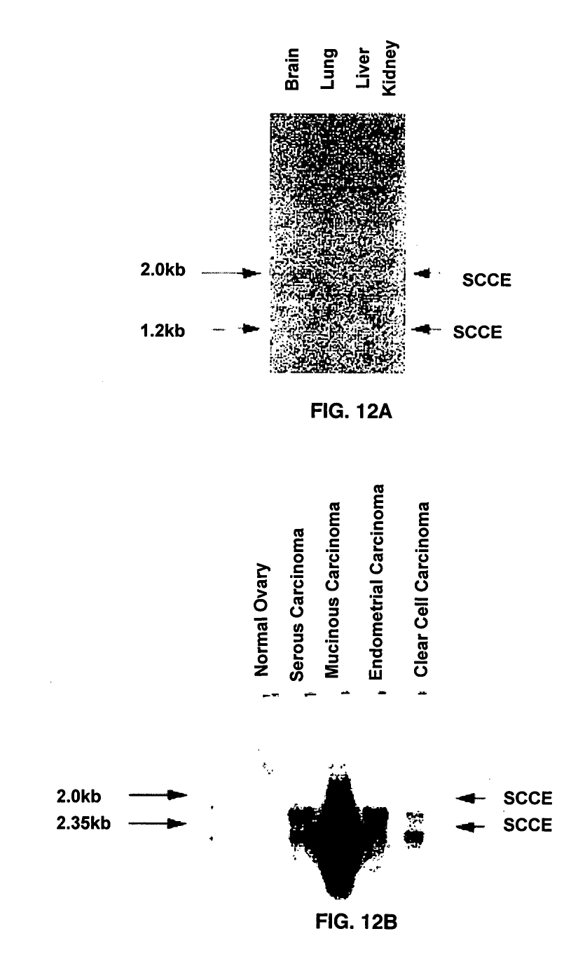

Northern blots were probed with the PCR product to determine expression of stratum corneum chymotrypsin enzyme in fetal tissue and ovarian carcinoma (FIGS. 12A & 12B). Noticeably, detection of stratum corneum chymotrypsin enzyme messenger RNA on the fetal Northern was almost non-existent (a problem with the probe or the blot was excluded by performing the proper controls). A faint band appeared in fetal kidney. On the other hand, stratum corneum chymotrypsin enzyme mRNA is abundant in the ovarian carcinoma mRNA (FIG. 12B). Two transcripts of the correct size are observed for stratum corneum chymotrypsin enzyme. The same panel of cDNA used for hepsin analysis was used for stratum corneum chymotrypsin enzyme expression.

No stratum corneum chymotrypsin enzyme expression was detected in the normal ovary lane of the Northern blot. A comparison of all candidate genes, including a loading marker (β-tubulin), was shown to confirm that this observation was not a result of a loading bias. Quantitative PCR using stratum corneum chymotrypsin enzyme primers, along with β-tubulin internal control primers, confirmed the overexpression of stratum corneum chymotrypsin enzyme mRNA in carcinoma of the ovary with no expression in normal ovarian tissue.

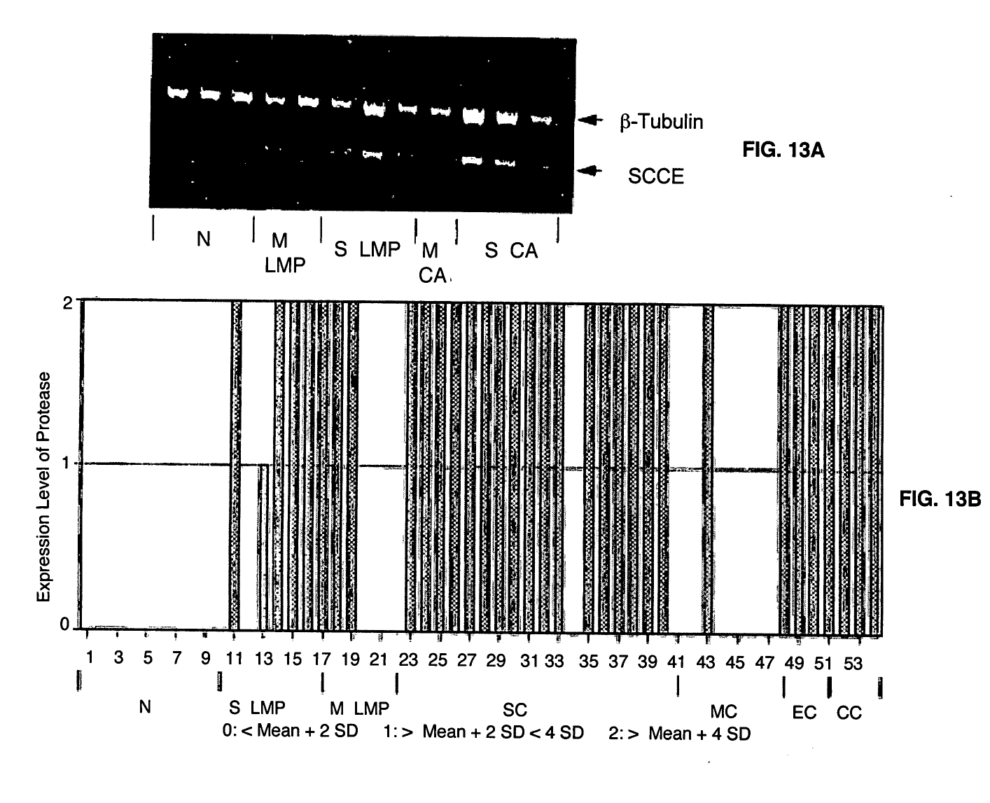

FIG. 13A shows a comparison using quantitative PCR of stratum corneum chymotrypsin enzyme cDNA from normal ovary and ovarian carcinomas. FIG. 13B shows the ratio of stratum corneum chymotrypsin enzyme to the β-tubulin internal standard in 10 normal and 44 ovarian carcinoma tissues. Again, it is observed that stratum corneum chymotrypsin enzyme is highly overexpressed in ovarian carcinoma cells. It is also noted that some mucinous tumors overexpress stratum corneum chymotrypsin enzyme, but the majority do not.

### Protease M

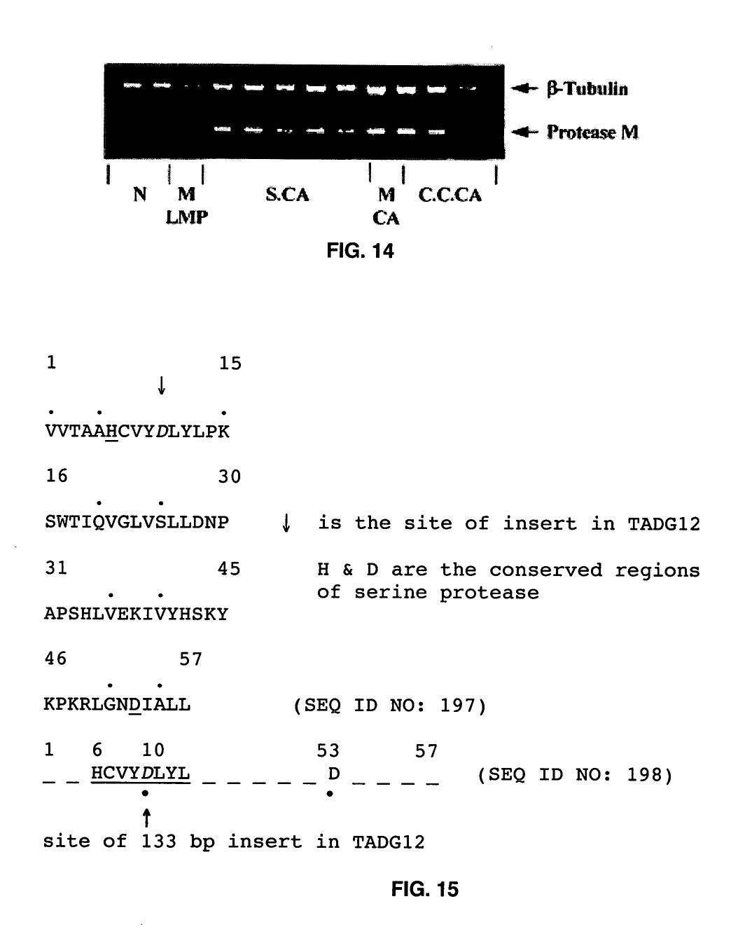

Protease M was identified from subclones of the His-ser primer pair. This protease was first cloned by Anisowicz, et al., [Molecular Medicine, 2, 624-636 (1996)] and shown to be overexpressed in carcinomas. A preliminary evaluation indicates that this enzyme is overexpressed in ovarian carcinoma (FIG. 14).

### Cofactor I and Complement Factor B

Several serine proteases associated with the coagulation pathway were also subcloned. Examination of normal and ovarian carcinomas by quantitative PCR for expression of these enzymes, it was noticeable that this mRNA was not clearly overexpressed in ovarian carcinomas when compared to normal ovarian tissue. It should be noted that the same panel of tumors was used for the evaluation of each candidate protease.

### TADG-12

TADG-12 was identified from the primer pairs, sense-His/antisense-Asp (see FIG. 1, Lanes 1 & 2). Upon subcloning both PCR products in lane 2, the 200 bp product had a unique protease-like sequence not included in GenBank. This 200 bp product contains many of the conserved amino acids common for the His-Asp domain of the family of serine proteins. The second and larger PCR product (300 bp) was shown to have a high degree of homology with TADG-12 (His-Asp sequence), but also contained approximately 100 bp of unique sequence. Synthesis of specific primers and the sequencing of the subsequent PCR products from three different tumors demonstrated that the larger PCR product (present in about 50% of ovarian carcinomas) includes an insert of about 100 bp near the 5′ end (and near the histidine) of the sequence. This insert may be a retained genomic intron because of the appropriate position of splice sites and the fact that the insert does not contain an open reading frame (see FIG. 15). This suggests the possibility of a splice site mutation which gives rise to retention of the intron, or a translocation of a sequence into the TADG-12 gene in as many as half of all ovarian carcinomas.

### TADG-13 and TADG-14

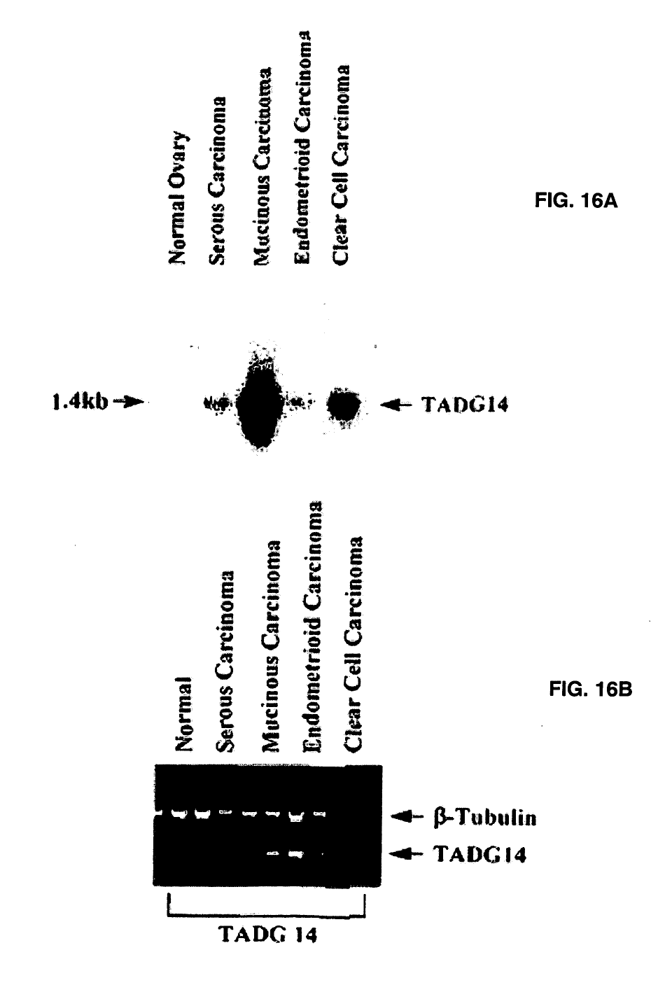

Specific primers were synthesized for TADG-13 and TADG-14 to evaluate expression of genes in normal and ovarian carcinoma tissue. Northern blot analysis of ovarian tissues indicates the transcript for the TADG-14 gene is approximately 1.4 kb and is expressed in ovarian carcinoma tissues (FIG. 16A) with no noticeable transcript presence in normal tissue. In quantitative PCR studies using specific primers, increased expression of TADG-14 in ovarian carcinoma tissues was noted compared to a normal ovary (FIG. 16B). The presence of a specific PCR product for TADG-14 in both an HeLa library and an ovarian carcinoma library was also confirmed. Several candidate sequences corresponding to TADG-14 have been screened and isolated from the HeLa library.

Clearly from sequence homology, these genes fit into the family of serine proteases. TADG-13 and TADG-14 are, however, heretofore undocumented genes which the specific primers of the invention allow to be evaluated in normal and tumor cells, and with which the presence or absence of expression of these genes is useful in the diagnosis or treatment selection for specific tumor types.

### PUMP-1

In a similar strategy using redundant primers to metal binding domains and conserved histidine domains, a differentially expressed PCR product identical to matrix metallo-protease 7 (MMP-7) was identified, herein called PUMP-1. Using specific primers for PUMP-1, PCR produced a 250 bp product for Northern blot analysis.

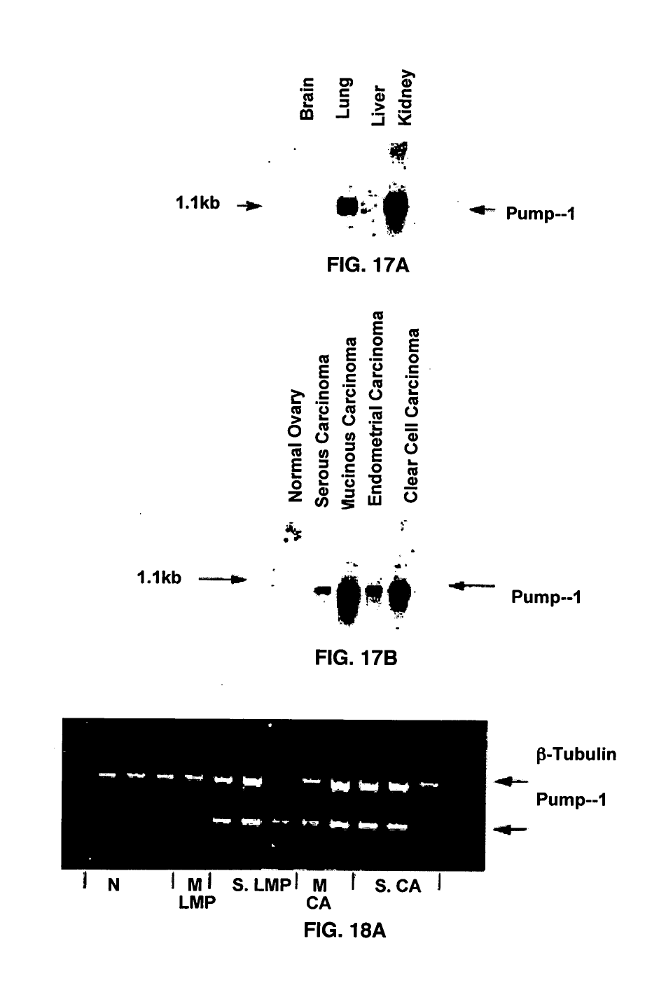

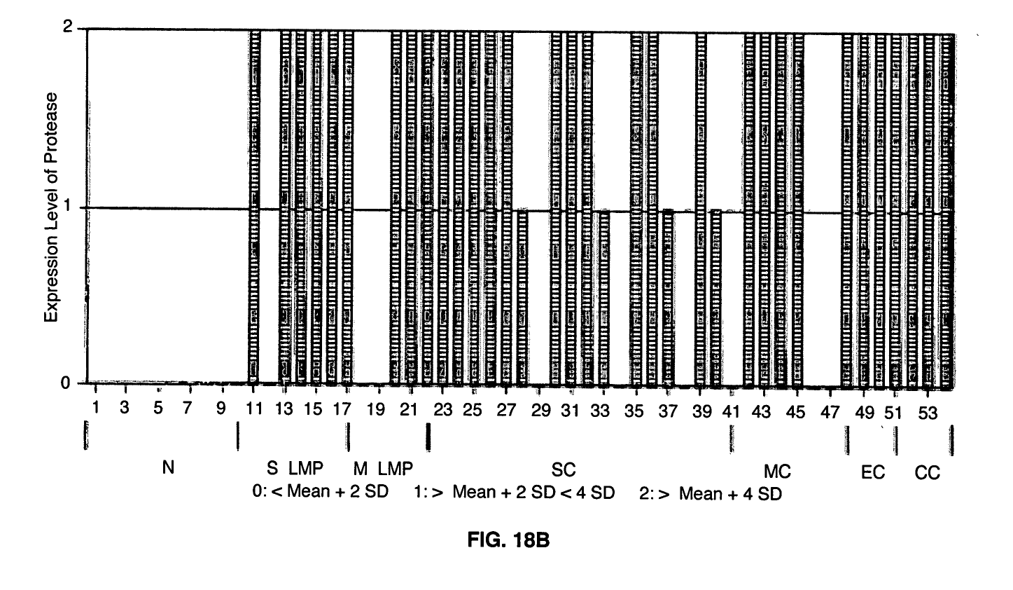

PUMP-1 is differentially expressed in fetal lung and kidney tissues. FIG. 17A shows the expression of PUMP-1 in human fetal tissue, while no transcript could be detected in either fetal brain or fetal liver. FIG. 17B compares PUMP-1 expression in normal ovary and carcinoma subtypes using Northern blot analysis. Notably, PUMP-1 is expressed in ovarian carcinoma tissues, and again, the presence of a transcript in normal tissue was not detected. Quantitative PCR comparing normal versus ovarian carcinoma expression of the PUMP-1 mRNA indicates that this gene is highly expressed in serous carcinomas, including most low malignant serous tumors, and is, again, expressed to a lesser extent in mucinous tumors (see FIGS. 18A & 18B). PUMP-1, however, is so far the protease most frequently found overexpressed in mucinous tumors (See Table 7).

### Cathepsin-L

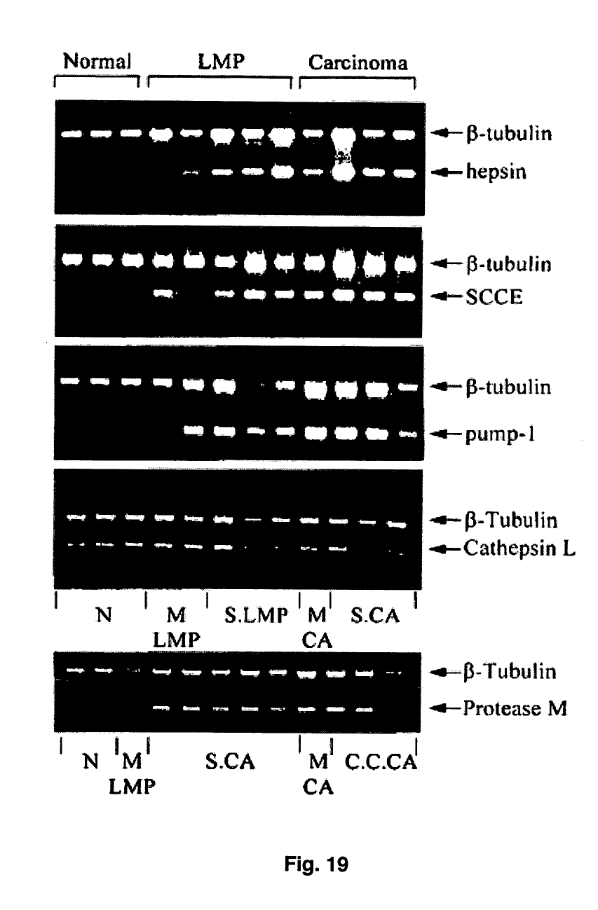

Using redundant cysteine protease primers to conserved domains surrounding individual cysteine and histidine residues, the cathepsin-L protease was identified in several serous carcinomas. An initial examination of the expression of cathepsin L in normal and ovarian tumor tissue indicates that transcripts for the cathepsin-L protease are present in both normal and tumor tissues (FIG. 19). However, its presence or absence in combination with other proteases of the present invention permits identification of specific tumor types and treatment choices.

### Discussion

Redundant primers to conserved domains of serine, metallo-, and cysteine proteases have yielded a set of genes whose mRNAs are overexpressed in ovarian carcinoma. The genes which are clearly overexpressed include the serine proteases hepsin, stratum corneum chymotrypsin enzyme, protease M TADG12, TADG14 and the metallo-protease PUMP-1 (see FIG. 19 and Table 7). Northern blot analysis of normal and ovarian carcinoma tissues, summarized in FIG. 14, indicated overexpression of hepsin, stratum corneum chymotrypsin enzyme, PUMP-1 and TADG-14. A β-tubulin probe to control for loading levels was included.

TABLE 7
Overexpression of Proteases in Ovarian Tumors

| Type | N | Hepsin | | SCCE | | Pump-1 | | Protease M | |
|---|---|---|---|---|---|---|---|---|---|
| Normal 10 | 0% | (0/10) | | 0% | (0/10) | 0% | (0/10) | 0% | (0/10) |
| LMP | 12 | 58.3% | (7/12) | 66.7% | (8/12) | 75.0% | (9/12) | 75% | (9/12) |
| serous | 7 | 85.7% | (6/7) | 85.7% | (6/7) | 85.7% | (6/7) | 100% | (7/7) |
| mucinous | 5 | 20.0% | (1/5) | 40.0% | (2/5) | 60% | (3/5) | 40.0% | (2/5) |
| Carcinoma | 32 | 84.4% | (27/32) | 78.1% | (25/32) | 81.3% | (26/32) | 90.6% | (29/32) |
| serous | 19 | 94.7% | (18/19) | 89.5% | (17/19) | 78.9% | (15/19) | 94.7% | (18/19) |
| mucinous | 7 | 42.9% | (3/7) | 28.6% | (2/7) | 71.4% | (5/7) | 85.7% | (6/7) |
| endometr. | 3 | 100% | (3/3) | 100% | (3/3) | 100% | (3/3) | 100% | (3/3) |
| clear cell | 3 | 100% | (3/3) | 100% | (3/3) | 100% | (3/3) | 67.7% | (2/3) |

For the most part, these proteins previously have not been associated with the extracellular matrix of ovarian carcinoma cells. No panel of proteases which might contribute to the growth, shedding, invasion and colony development of metastatic carcinoma has been previously described, including the three new candidate serine proteases which are herein disclosed. The establishment of an extracellular protease panel associated with either malignant growth or malignant potential offers the opportunity for the identification of diagnostic or prognostic markers and for therapeutic intervention through inhibition or down regulation of these proteases.

The availability of the instant gene-specific primers coding for the appropriate region of tumor specific proteases allows for the amplification of a specific cDNA probe using Northern and Southern analysis, and their use as markers to detect the presence of the cancer in tissue. The probes also allow more extensive evaluation of the expression of the gene in normal ovary versus low malignant potential tumor, as well as both high- and low-stage carcinomas. The evaluation of a panel of fresh frozen tissue from all the carcinoma subtypes (Table 4) allowed the determination of whether a protease is expressed predominantly in early stage disease or within specific carcinoma subtypes. It was also determined whether each gene's expression is confined to a particular stage in tumor progression and/or is associated with metastatic lesions. Detection of specific combinations of proteases is an identifying characteristic of the specific tumor types and yields valuable information for diagnoses and treatment selection. Particular tumor types may be more accurately diagnosed by the characteristic expression pattern of each specific tumor.

### EXAMPLE 10

### Hepsin Peptide Ranking

For vaccine or immune stimulation, individual 9-mers to 11-mers of the hepsin protein were examined to rank the binding of individual peptides to the top 8 haplotypes in the general population. The computer program used for this analyses can be found on the web site of National Institutes of Health. Table 8 shows the peptide ranking based upon the predicted half-life of each peptide's binding to a particular HLA allele. A larger half-life indicates a stronger association with that peptide and the particular HLA molecule. The hepsin peptides that strongly bind to an HLA allele are putative immunogens, and are used to innoculate an individual against hepsin.

Hepsin peptide ranking

| HLA Type | | | Predicted | SEQ |
|---|---|---|---|---|
| & Ranking | Start | Peptide | Dissociation_{1/2} | ID NO: |
| HLA A0201 | | | | |
| 1 | 170 | SLGRWPWQV | 521.640 | 28 |
| 2 | 191 | SLLSGDWVL | 243.051 | 29 |
| 3 | 229 | GLQLGVQAV | 159.970 | 30 |
| 4 | 392 | KVSDFREWI | 134.154 | 31 |
| 5 | 308 | VLQEARVPI | 72.717 | 32 |
| 6 | 130 | RLLEVISVC | 71.069 | 33 |
| 7 | 98 | ALTHSELDV | 69.552 | 34 |
| 8 | 211 | VLSRWRVFA | 46.451 | 35 |
| 9 | 26 | LLLLTAIGA | 31.249 | 36 |
| 10 | 284 | ALVDGKICT | 30.553 | 37 |
| 11 | 145 | FLAAICQDC | 22.853 | 38 |
| 12 | 192 | LLSGDWVLT | 21.536 | 39 |
| 13 | 20 | ALTAGTLLL | 21.362 | 40 |
| 14 | 259 | ALVHLSSPL | 21.362 | 41 |
| 15 | 277 | CLPAAGQAL | 21.362 | 42 |
| 16 | 230 | LQLGVQAVV | 18.186 | 43 |
| 17 | 268 | PLTEYIQPV | 14.429 | 44 |
| 18 | 31 | AIGAASWAI | 10.759 | 45 |
| 19 | 285 | LVDGKICTV | 9.518 | 46 |
| 20 | 27 | LLLTAIGAA | 9.343 | 47 |
| HLA A0205 | | | | |
| 1 | 191 | SLLSGDWVL | 25.200 | 48 |
| 2 | 163 | IVGGRDTSL | 23.800 | 49 |
| 3 | 392 | KVSDFREWI | 18.000 | 50 |
| 4 | 64 | MVFDKTEGT | 15.300 | 51 |
| 5 | 236 | AVVYHGGYL | 14.000 | 52 |
| 6 | 55 | QVSSADARL | 14.000 | 53 |
| 7 | 130 | RLLEVISVC | 9.000 | 54 |
| 8 | 230 | LQLGVQAVV | 8.160 | 55 |
| 9 | 20 | ALTAGTLLL | 7.000 | 56 |
| 10 | 259 | ALVHLSSPL | 7.000 | 57 |
| 11 | 277 | CLPAAGQAL | 7.000 | 58 |
| 12 | 17 | KVAALTAGT | 6.000 | 59 |
| 13 | 285 | LVDGKICTV | 5.440 | 60 |
| 14 | 308 | VLQEARVPI | 5.100 | 61 |
| 15 | 27 | LLLTAIGAA | 5.100 | 62 |
| 16 | 229 | GLQLGVQAV | 4.000 | 63 |
| 17 | 313 | RVPIISNDV | 4.000 | 64 |
| 18 | 88 | LSCEEMGFL | 3.570 | 65 |
| 19 | 192 | LLSGDWVLT | 3.400 | 66 |
| 20 | 284 | ALVDGKICT | 3.000 | 67 |
| HLA A1 | | | | |
| 1 | 89 | SCEEMGFLR | 45.000 | 68 |
| 2 | 58 | SADARLMVF | 25.000 | 69 |
| 3 | 393 | VSDFREWIF | 7.500 | 70 |
| 4 | 407 | HSEASGMVT | 6.750 | 71 |
| 5 | 137 | VCDCPRGRF | 5.000 | 72 |
| 6 | 269 | LTEYIQPVC | 4.500 | 73 |
| 7 | 47 | DQEPLYPVQ | 2.700 | 74 |
| 8 | 119 | CVDEGRLPH | 2.500 | 75 |
| 9 | 68 | KTEGTWRLL | 2.250 | 76 |
| 10 | 101 | HSELDVRTA | 1.350 | 77 |
| 11 | 250 | NSEENSNDI | 1.350 | 78 |
| 12 | 293 | VTGWGNTQY | 1.250 | 79 |
| 13 | 231 | QLGVQAVVY | 1.000 | 80 |
| 14 | 103 | ELDVRTAGA | 1.000 | 81 |
| 15 | 378 | GTGCALAQK | 1.000 | 82 |
| 16 | 358 | VCEDSISRT | 0.900 | 83 |
| 17 | 264 | SSPLPLTEY | 0.750 | 84 |
| 18 | 87 | GLSCEEMGF | 0.500 | 85 |
| 19 | 272 | YIQPVCLPA | 0.500 | 86 |
| 20 | 345 | GIDACQGDS | 0.500 | 87 |
| HLA A24 | | | | |
| 1 | 301 | YYGQQAGVL | 200.000 | 88 |
| 2 | 238 | VYHGGYLPF | 100.000 | 89 |
| 3 | 204 | CFPERNRVL | 36.000 | 90 |
| 4 | 117 | FFCVDEGRL | 20.000 | 91 |
| 5 | 124 | RLPHTQRLL | 12.000 | 92 |
| 6 | 80 | RSNARVAGL | 12.000 | 93 |
| 7 | 68 | KTEGTWRLL | 12.000 | 94 |
| 8 | 340 | GYPEGGIDA | 9.000 | 95 |
| 9 | 242 | GYLPFRDPN | 9.000 | 96 |
| 10 | 51 | LYPVQVSSA | 7.500 | 97 |
| 11 | 259 | ALVHLSSPL | 7.200 | 98 |
| 12 | 277 | CLPAAGQAL | 7.200 | 99 |
| 13 | 191 | SLLSGDWVL | 6.000 | 100 |
| 14 | 210 | RVLSRWRVF | 6.000 | 101 |
| 15 | 222 | VAQASPHGL | 6.000 | 102 |
| 16 | 236 | AVVYHGGYL | 6.000 | 103 |
| 17 | 19 | AALTAGTLL | 6.000 | 104 |
| 18 | 36 | SWAIVAVLL | 5.600 | 105 |
| 19 | 35 | ASWAIVAVL | 5.600 | 106 |
| 20 | 300 | QYYGQQAGV | 5.600 | 107 |
| HLA B7 | | | | |
| 1 | 363 | ISRTPRWRL | 90.000 | 108 |
| 2 | 366 | TPRWRLCGI | 80.000 | 109 |
| 3 | 236 | AVVYHGGYL | 60.000 | 110 |
| 4 | 13 | CSRPKVAAL | 40.000 | 111 |
| 5 | 179 | SLRYDGAHL | 40.000 | 112 |
| 6 | 43 | LLRSDQEPL | 40.000 | 113 |
| 7 | 19 | AALTAGTLL | 36.000 | 114 |
| 8 | 55 | QVSSADARL | 20.000 | 115 |
| 9 | 163 | IVGGRDTSL | 20.000 | 116 |
| 10 | 140 | CPRGRFLAA | 20.000 | 117 |
| 11 | 20 | ALTAGTLLL | 12.000 | 118 |
| 12 | 409 | EASGMVTQL | 12.000 | 119 |
| 13 | 259 | ALVHLSSPL | 12.000 | 120 |
| 14 | 35 | ASWAIVAVL | 12.000 | 121 |
| 15 | 184 | GAHLCGGSL | 12.000 | 122 |
| 16 | 18 | VAALTAGTL | 12.000 | 123 |
| 17 | 222 | VAQASPHGL | 12.000 | 124 |
| 18 | 224 | QASPHGLQL | 12.000 | 125 |
| 19 | 265 | SPLPLTEYI | 8.000 | 126 |
| 20 | 355 | GPFVCEDSI | 8.00 | 127 |
| HLA B8 | | | | |
| 1 | 13 | CSRPKVAAL | 80.000 | 128 |
| 2 | 366 | TPRWRLCGI | 80.000 | 129 |
| 3 | 140 | CPRGRFLAA | 16.000 | 130 |
| 4 | 152 | DCGRRKLPV | 4.800 | 131 |
| 5 | 363 | ISRTPRWRL | 4.000 | 132 |
| 6 | 163 | IVGGRDTSL | 4.000 | 133 |
| 7 | 331 | QIKPKMFCA | 4.000 | 134 |
| 8 | 80 | RSNARVAGL | 2.000 | 135 |
| 9 | 179 | SLRYDGAHL | 1.600 | 136 |
| 10 | 43 | LLRSDQEPL | 1.600 | 137 |
| 11 | 409 | EASGMVTQL | 1.600 | 138 |
| 12 | 311 | EARVPIISN | 0.800 | 139 |
| 13 | 222 | VAQASPHGL | 0.800 | 140 |
| 14 | 19 | AALTAGTLL | 0.800 | 141 |
| 15 | 18 | VAALTAGTL | 0.800 | 142 |
| 16 | 184 | GAHLCGGSL | 0.800 | 143 |
| 17 | 224 | QASPHGLQL | 0.800 | 144 |
| 18 | 82 | NARVAGLSC | 0.800 | 145 |
| 19 | 204 | CFPERNRVL | 0.600 | 146 |
| 20 | 212 | LSRWRVFAG | 0.400 | 147 |
| HLA B2702 | | | | |
| 1 | 172 | GRWPWQVSL | 300.000 | 148 |
| 2 | 44 | LRSDQEPLY | 200.00 | 149 |
| 3 | 155 | RRKLPVDRI | 180.000 | 150 |
| 4 | 213 | SRWRVFAGA | 100.000 | 151 |
| 5 | 166 | GRDTSLGRW | 100.000 | 152 |
| 6 | 369 | WRLCGIVSW | 100.000 | 153 |
| 7 | 180 | LRYDGAHLC | 100.000 | 154 |
| 8 | 96 | LRALTHSEL | 60.000 | 155 |
| 9 | 396 | FREWIFQAI | 60.000 | 156 |
| 10 | 123 | GRLPHTQRL | 60.000 | 157 |
| 11 | 207 | ERNRVLSRW | 30.000 | 158 |
| 12 | 209 | NRVLSRWRV | 20.000 | 159 |
| 13 | 14 | SRPKVAALT | 20.000 | 160 |
| 14 | 106 | VRTAGANGT | 20.000 | 161 |
| 15 | 129 | QRLLEVISV | 20.000 | 162 |
| 16 | 349 | CQGDSGGPF | 20.000 | 163 |
| 17 | 61 | ARLMVFDKT | 20.000 | 164 |
| 18 | 215 | WRVFAGAVA | 20.000 | 165 |
| 19 | 143 | GRFLAAICQ | 10.000 | 166 |
| 20 | 246 | FRDPNSEEN | 10.000 | 167 |
| HLA B4403 | | | | |
| 1 | 132 | LEVISVCDC | 36.000 | 168 |
| 2 | 91 | EEMGFLRAL | 18.000 | 169 |
| 3 | 264 | SSPLPLTEY | 13.500 | 170 |
| 4 | 310 | QEARVPIIS | 12.000 | 171 |
| 5 | 319 | NDVCNGADF | 10.000 | 172 |
| 6 | 4 | KEGGRTVPC | 9.000 | 173 |
| 7 | 251 | SEENSNDIA | 8.000 | 174 |
| 8 | 256 | NDIALVHLS | 7.500 | 175 |
| 9 | 294 | TGWGNTQYY | 6.750 | 176 |
| 10 | 361 | DSISRTPRW | 6.750 | 177 |
| 11 | 235 | QAVVYHGGY | 6.000 | 178 |
| 12 | 109 | AGANGTSGF | 6.000 | 179 |
| 13 | 270 | TEYIQPVCL | 6.000 | 180 |
| 14 | 174 | WPWQVSLRY | 4.500 | 181 |
| 15 | 293 | VTGWGNTQY | 4.500 | 182 |
| 16 | 69 | TEGTWRLLC | 4.000 | 183 |
| 17 | 90 | CEEMGFLRA | 4.000 | 184 |
| 18 | 252 | EENSNDIAL | 4.000 | 185 |
| 19 | 48 | QEPLYPVQV | 4.000 | 186 |
| 20 | 102 | SELDVRTAG | 3.600 | 187 |

### EXAMPLE 11

### Hepsin Peptides as Target Epitopes for Human CD8⁺ Cytotoxic T Cells

Two computer programs were used to identify 9-mer peptides containing binding motifs for HLA class I molecules. The first, based on a scheme devised by Parker et al. (6), was developed by the Bioinformatics and Molecular Analysis Section (BIMAS) of the Center for Information Technology, NIH, and the second, known as SYFPEITHI, was formulated by Rammensee and colleagues at the University of Tubingen, Germany.

Peptides that possessed HLA A2.1 binding motifs were synthesized and tested directly for their ability to bind HLA A2.1. This technique employs T2 cells which are peptide transporter-deficient and thus express low endogenous HLA class I levels due to inability to load peptide and stabilize HLA class I folding for surface expression. It has been showed that addition of exogenous peptides capable of binding HLA A2.1 (A*0201) could increase the number of properly folded HLA A2.1 molecules on the cell surface, as revealed by flow cytometry (7).

Peptides that possessed binding motifs for HLA class I molecules other than A2.1 were tested directly for their ability to induce specific CD8⁺ CTL responses from normal adult donors as described below.

Monocyte-derived DC were generated from peripheral blood drawn from normal adult donors of the appropriate HLA type. Adherent monocytes were cultured in AIM-V (Gibco-BRL) supplemented with GM-CSF and IL-4 according to standard techniques (8-9). After 5-6 days, DC maturation was induced by addition of PGE₂, IL-1 b and TNFa for a further 48 h.

Mature DC were loaded with peptide (2×10⁶ DC with 50 mg/ml peptide in 1 ml serum-free AIM-V medium for 2 h at 37° C.) and washed once prior to culture with 1×10⁶/ml peripheral blood mononuclear cells (PBMC) in AIM-V or AIM-V plus 5% human AB serum. The PBMC:DC ratio was between 20:1 and 30:1. After 7 days, responder T cells were restimulated with peptide-loaded, irradiated autologous DC or PBMC at responder:stimulator ratios between 10:1 and 20:1 or 1:1 and 1:10 respectively. At this point, cultures were supplemented with recombinant human IL-2 (10-100 U/ml), and fed with 50-75% changes of fresh medium plus IL-2 every 2-4 days. T cell lines were established and maintained by peptide restimulation every 14-21 days. Responder CD8⁺ T cells were purified by positive selection with anti-CD8-coupled magnetic beads (Dynal, Inc.) after the 2^{nd} or 3^{rd} antigen stimulation.

Peptide-specific cytotoxicity was tested in standard 5-6 h microwell ⁵¹Cr-release assays (10). Autologous EBV-transformed lymphoblastoid cell lines (LCL) were loaded with peptide (50 mg/ml, 1 h at 37° C.) and subsequently ⁵¹Cr-labeled (50 mCi in 200-300 ml, 1 h at 37° C.). Peptide-loaded ⁵¹Cr-labeled LCL were incubated with CD8⁺ T cells at effector-target ration between 10:1 and 1.25:1. Cytotoxicity was recorded as percentage ⁵¹Cr released into culture supernatants.

### Hepsin Peptide 170-178

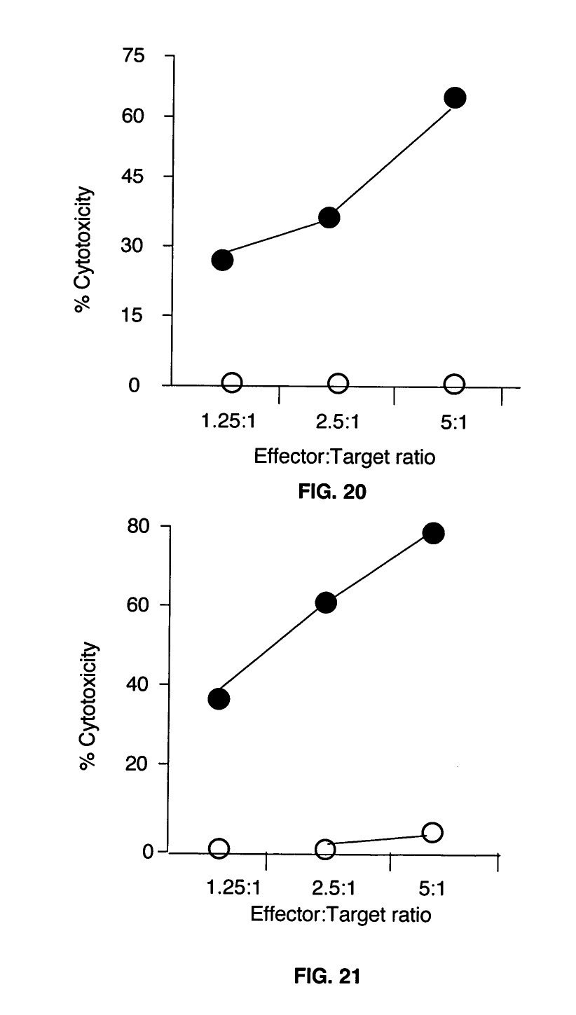

Hepsin peptide 170-178 (SEQ ID NO: 28) is an HLA A2.1-binding peptide, as revealed by upregulation of A2.1 expression in T2 cells (data not shown). CD8⁺ CTL specific for hepsin 170-178 killed peptide-loaded autologous LCL, but did not kill control, peptide-free LCL (FIG. 20). Heterologous HLA A2.1-expressing peptide-loaded LCL were efficiently killed, but targets lacking HLA A2.1 were not killed. Natural killer-sensitive K562 cells were not lysed. Cytotoxicity against hepsin 170-178 loaded LCL could be blocked with MAb specific for a non-polymorphic HLA class I determinant, confirming that lysis was HLA class I-restricted. Cytotoxicity was also blocked by MAb specific for HLA A2.1

### Hepsin Peptide 172-180

Hepsin peptide 172-180 (SEQ ID NO: 148) was predicted by computer analysis to bind HLA B27. While this could not be demonstrated directly, cytotoxicity assays showed that CD8⁺ CTL specific for hepsin 172-180 could kill peptide-loaded, HLA B27-expressing autologous and heterologous LCL, but failed to recognize heterologous peptide-loaded LCL that did not express HLA B27, or peptide-free control LCL (FIG. 21). Natural killer-sensitive K562 cells were not lysed. Cytotoxicity against hepsin 172-180 loaded LCL could be blocked with MAb specific for a non-polymorphic HLA class I determinant, confirming that lysis was HLA class I-restricted.

### Hepsin Peptide 42-51

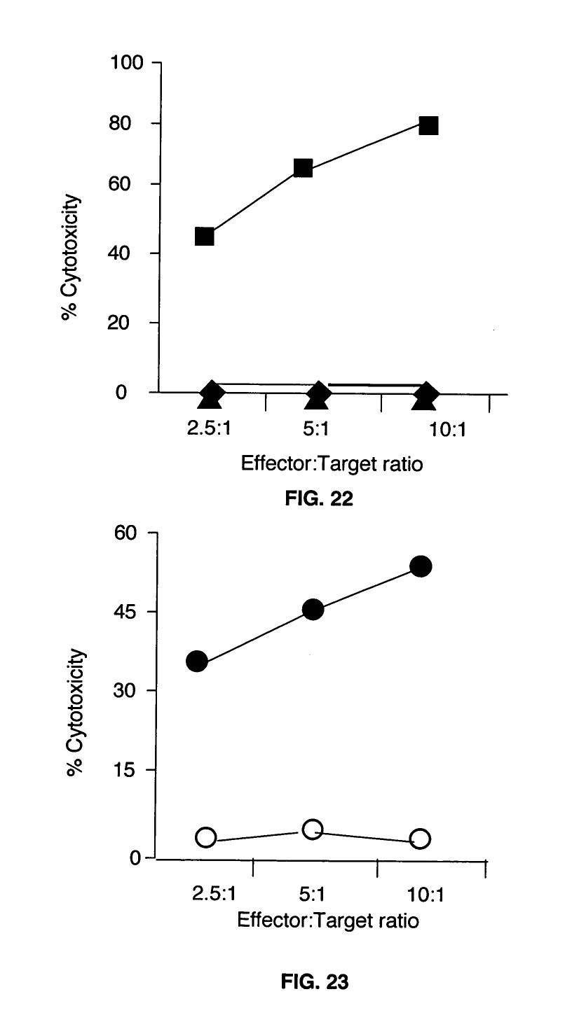

Hepsin peptide 42-51 (SEQ ID NO: 189) was predicted by computer analysis to bind HLA A*0201. CD8⁺ CTL specific for hepsin 42-51 killed peptide-loaded autologous LCL, but did not kill control, peptide-free LCL (FIG. 22). Heterologous HLA A*0201-expressing peptide-loaded LCL were efficiently killed, but targets lacking HLA A*0201 were not killed. Natural killer-sensitive K562 cells were not lysed. Cytotoxicity against hepsin 42-51 loaded LCL could be blocked with MAb specific for a non-polymorphic HLA class I determinant, confirming that lysis was HLA class I-restricted. Cytotoxicity was also blocked by MAb specific for HLA A2.1

### Hepsin Peptide 284-293

Hepsin peptide 284-293 (SEQ ID NO: 190) was predicted by computer analysis to bind HLA A*0201. CD8⁺ CTL specific for hepsin 284-293 killed peptide-loaded autologous LCL, but did not kill control, peptide-free LCL (FIG. 23). Heterologous HLA A*0201-expressing peptide-loaded LCL were efficiently killed, but targets lacking HLA A*0201 were not killed. Natural killer-sensitive K562 cells were not lysed. Cytotoxicity against hepsin 284-293 loaded LCL could be blocked with MAb specific for a non-polymorphic HLA class I determinant, confirming that lysis was HLA class I-restricted.

### Hepsin Peptide 308-317

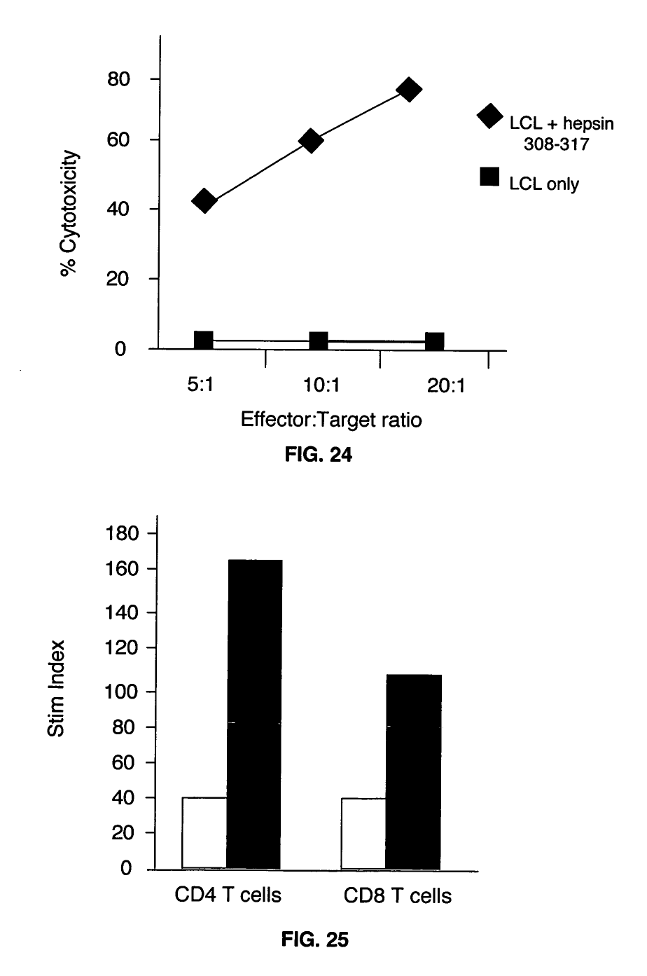

Hepsin peptide 308-317 (SEQ ID NO: 191) was predicted by computer analysis to bind HLA A*0201. CD8⁺ CTL specific for hepsin 308-317 killed peptide-loaded autologous LCL, but did not kill control, peptide-free LCL (FIG. 24). Heterologous HLA A*0201-expressing peptide-loaded LCL were efficiently killed, but targets lacking HLA A*0201 were not killed. Cytotoxicity against hepsin 308-317 loaded LCL could be blocked with MAb specific for a non-polymorphic HLA class I determinant, confirming that lysis was HLA class I-restricted.

### EXAMPLE 12

### Recombinant Full Length Hepsin Induces CD4⁺ and CD8⁺ T Cell Proliferative Responses

Results disclosed above that show dendritic cells (DC) loaded with hepsin-derived peptides can efficiently stimulate HLA A2.1-restricted and HLA B27-restricted CD8⁺ CTL responses in normal adults suggest that hepsin may be a leading candidate as a target for dendritic cell-based immunotherapy of ovarian cancer. Furthermore, the utility of hepsin as a target antigen for immunotherapeutic purposes may not be confined to ovarian cancer. A recent series of gene expression profiling studies identified hepsin as a major tumor marker for prostate cancer. Hepsin was consistently highly expressed in prostate cancer, but not in benign prostatic hyperplasia (11-15). These reports strongly support the proposal that hepsin may also be a leading target for dendritic cell-based immunotherapy of prostate cancer.

However, the use of peptides restricts the response to predetermined HLA class I types, which imposes limitations on patient selection. The use of dendritic cells loaded with full-length recombinant tumor antigens circumvents this problem, and also offers the prospect of being able to induce both CD8⁺ T cell responses and helper CD4⁺ T cell responses, the latter of which may play a critical role in the generation and maintenance of effective anti-tumor immunity. A further, potentially critical, advantage of using full-length tumor antigen is that CD8⁺ T cell responses are induced against naturally processed epitopes, which markedly increases the likelihood that CD8⁺ T cells will recognize endogenously synthesized antigens that are also naturally processed and presented by the target ovarian tumor cell.

The following example shows that dendritic cells loaded with full-length recombinant hepsin are capable of inducing both CD4⁺ T cell and CD8⁺ T cell proliferative responses to hepsin.

Hepsin cDNA was cloned into the IPTG-inducible pQE-30 vector (Qiagen) and expressed in E. coli. Addition of a 6×-histidine tag on the amino terminus facilitates affinity purification with Ni-NTA resin. Dendritic cells were derived from peripheral blood monocyte precursors as described above. Mature dendritic cells express high levels of HLA class I and class II molecules, costimulatory molecules (e.g., CD86 and CD40), and CD83 (expressed on mature, but not immature, monocyte-derived dendritic cells), but do not express CD14 (a macrophage/monocyte marker).

To stimulate hepsin-specific T cell proliferation, mature dendritic cells were loaded with purified recombinant hepsin by DOTAP lipofection. Briefly 25 mg hepsin was combined with 15 mg DOTAP (Roche Applied Science, Indianapolis, Ind.) in 500 ml AIM-V medium (Invitrogen, Grand Island, N.Y.). This mixture was incubated with 1-2×10⁶ dendritic cells for up to 2 hours at 37° C. Hepsin-loaded dendritic cells were cocultured with autologous peripheral blood lymphocytes from a normal male donor at a responder:stimulator, ratio of 30:1 in AIM-V medium plus 5% human AB serum. After 7-10 days, responder T cells were restimulated with hepsin-loaded dendritic cells at a responder:stimulator ratio of 10:1. T cell cultures were supplemented with recombinant IL-2 (10-100 U/ml), and fed every 2-4 days with 50-75% changes of medium plus IL-2. T cell lines were subsequently maintained by restimulation with hepsin-loaded DC every 14 days. Before the 3^{rd} restimulation, CD4⁺ T cells and CD8⁺ T cells were purified by positive selection with anti-CD4 or anti-CD8-conjugated magnetic beads, as appropriate. Resultant populations were >98% pure by flow cytometry.

CD4⁺ T cells and CD8⁺ T cells were tested in microwell lymphoproliferation assays after the 4^{th} and 5^{th} passages, respectively. T cells (2×10⁴/well) were incubated with dendritic cells loaded with hepsin by DOTAP lipofection (5×10³/well) or control dendritic cells treated with DOTAP only (5×10³/well). The assay was incubated for 72 hours. Proliferation was determined by the addition of ³H-thymidine (1 mCi/well) to each microwell culture for the final 24 hours. Results are presented as the mean of triplicate microwells, calculated as a stimulaton index (ratio of ³H-thymidine uptake by T cells cultured with dendritic cells versus ³H-thymidine uptake by T cells cultured alone).

Although some background proliferation in response to stimulation with control dendritic cells was seen, this assay clearly shows that hepsin-loaded dendritic cells are capable of inducing a significant antigen-specific lymphoproliferative response by both CD4⁺ T cells and CD8⁺ T cells (FIG. 25). These results underline the potential for dendritic cell-based immunotherapy using hepsin as a tumor target antigen.

In summary, the present invention provides immunotherapeutic applications specially targeted at hepsin, and applied to the treatment of tumors that express hepsin. Target diseases will include ovarian cancer and prostate cancer, but will also include any other malignancy for which hepsin expression can be demonstrated. Immunotherapeutic applications will include, but are not limited to: Immunotherapy may take the form of hepsin-loaded dendritic cell vaccination, in which dendritic cells are generated in vitro from peripheral blood drawn from the patient, loaded with hepsin by lipofection or other means, and then given back to the patient as an autologous cellular vaccine, either in single doses or multiple doses. Hepsin may also be expressed in dendritic cells following transduction with a recombinant DNA vector, and such hepsin-transduced dendritic cells may then be used as a cellular vaccine.

Recombinant DNA vectors that express hepsin, either alone or as a fusion protein with other immunologically active components, may be used as a DNA vaccine for treatment of tumors that express hepsin. Hepsin-loaded or hepsin-expressing dendritic cells may also be used to stimulate tumor antigen-specific T cell responses in vitro, followed by adoptive immunotherapy, in which the patient will be given autologous hepsin-specific T cells.

Monoclonal antibody therapy based on hepsin are also apparent. Hepsin is expressed as a transmembrane protein on the surface of tumor cells. Construction of human monoclonal antibodies, or chimeric humanized monoclonal antibodies specific for hepsin offers an attractive option for immunotherapy of hepsin-expressing malignancies.

The following references were cited herein:
1. Torres-Rosedo et al. Proc. Natl. Acad. Sci. USA. 90, 7181-7185 (1993).
2. Powell et al. Cancer Research, 53, 417-422 (1993).
3. Noonan et al. Proc. Natl. Acad. Sci. USA, 87:7160-7164 (1990).
4. Leytus et al. [Biochemistry, 27, 1067-1074 (1988)]
5. Hall et al. [Mol. Cell. Biol., 3, 854-862 (1983)]
6. Parker et al. J. Immunol. 152:163-175 (1994).
7. Nimjin et al. Eur. J. Immunol. 23:1215-1219 (1993).
8. Santin et al. Obstetrics & Gynecology 96:422-430 (2000).
9. Santin et al. Am. J. Obstet. Gynecol. 183:601-609 (2001).
10. Nazaruk et al. Blood 91:3875-3883 (1998).
11. Luo et al. Cancer Res. 61:4683-4688 (2001).
12. Magee et al. Cancer Res. 61:5692-5696 (2001).
13. Welsh et al. Cancer Res. 61:5974-5978 (2001).
14. Dhanasekaran et al. Nature 412:822-826 (2001).
15. Stamey et al. J. Urol. 166:2171-2177 (2001).

Any patents or publications mentioned in this specification are indicative of the levels of those skilled in the art to which the invention pertains. Further, these patents and publications are incorporated by reference herein to the same extent as if each individual publication was specifically and individually incorporated by reference.

One skilled in the art will appreciate readily that the present invention is well adapted to carry out the objects and obtain the ends and advantages mentioned, as well as those objects, ends and advantages inherent herein. The present examples, along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. An immunogenic composition, comprising:
a full length hepsin protein or a fragment of a hepsin protein; and
an appropriate adjuvant.

2. The immunogenic composition of claim 1, wherein the length of said hepsin fragment is from 9-residue long to 20-residue long.

3. The immunogenic composition of claim 2, wherein said fragment is selected from the group consisting of SEQ ID NOS: 28, 29, 30, 31, 88, 89, 108, 109, 128, 129, 148, 149, 150, 151, 152, 153, 154, 189, 190, and 191.

4. A method of immunotherapy targeted toward hepsin in an individual, comprising the steps of:
a) isolating dendritic cells from the individual;
b) expressing or introducing a hepsin protein or peptide thereof comprising the immunogenic composition of claim 34 in the dendritic cells; and
c) transferring the dendritic cells back to the individual, wherein the dendritic cells would activate hepsin-specific immune responses in the individual, thereby generating immunotherapy targeted toward hepsin in said individual.

5. The method of claim 4, further comprising the steps of:
d) exposing immune cells comprising T cells isolated from the individual to the dendritic cells expressing or having the hepsin protein or peptide thereof, said dendritic cells generating hepsin-specific T cells from the immune cells; and
e) transferring the immune cells back to the individual, wherein said immune cells activate hepsin-specific immune responses in the individual, thereby generating immunotherapy targeted toward hepsin therein.

6. The method of claim 24, wherein expression or introduction of the hepsin protein or peptide thereof in the dendritic cells is obtained by transfection, transduction and loading the dendritic cells with the hepsin protein or peptide thereof.

7. A method of producing immune-activated cells directed toward hepsin, comprising the step of:
loading immune cells with the hepsin protein or peptide thereof comprising the immunogenic composition of claim 1, said hepsin protein or peptide thereof activating said immune cells, thereby producing immune-activated cells directed toward hepsin.

8. The method of claim 7, wherein the immune cells are isolated from an individual prior to said loading step, the method further comprising reintroducing the hepsin protein- or hepsin peptide-loaded immune cells into the individual subsequent to loading.

9. The method of claim 7, wherein the immune cells are B cells, T cells and dendritic cells.

10. A method of vaccinating an individual against hepsin, comprising the step of:
inoculating an individual with an expression vector encoding the hepsin protein or peptide thereof comprising the immunogenic composition of claim 1, wherein expression of said hepsin protein or peptide thereof elicits an immune response in the individual, thereby vaccinating said individual against hepsin.

11. A method of inhibiting hepsin protein in a cell, comprising the step of:
introducing into the cell an antibody which is specific for the hepsin protein or peptide thereof comprising the immunogenic composition of claim 1, wherein binding of the antibody to the hepsin protein or peptide thereof inhibits hepsin protein in the cell.

12. The method of claim 11, said antibody further comprising a therapeutic moiety.

13. The method of claim 12, wherein the therapeutic moiety is a radioisotope, a toxin, a chemotherapeutic agent, an immune stimulant, or a cytotoxic agent.

14. The method of claim 1, wherein the individual has, is suspected of having or is at risk of getting ovarian cancer, lung cancer, prostate cancer, or colon cancer.

15. An oligonucleotide having a sequence complementary to SEQ ID NO:188.

16. A composition comprising the oligonucleotide of claim 15 and a physiologically acceptable carrier.

17. A method of inhibiting expression of endogenous hepsin in a cell, comprising the step of:
introducing into the cell a vector comprising the oligonucleotide of claim 15 operably linked to elements necessary for expression, wherein expression of the vector in the cell produces hepsin antisense mRNA that hybridizes to endogenous hepsin mRNA, thereby inhibiting expression of endogenous hepsin in the cell.

18. A method of treating a neoplastic state in an individual in need of such treatment, comprising the step of:
administering to the individual an effective dose of the oligonucleotide of claim 15.

19. The method of claim 18, wherein said neoplastic state is selected from the group consisting of ovarian cancer, breast cancer, lung cancer, colon cancer and prostate cancer.

20. A method of screening for compounds that inhibit hepsin activity, comprising the steps of:
(a) contacting a sample comprising hepsin protein with a compound; and
(b) assaying for hepsin protease activity, wherein a decrease in said hepsin protease activity in the presence of said compound relative to hepsin protease activity in the absence of said compound indicates said compound inhibits hepsin activity.
